SEQUENCE LISTING (3 sequences)

SEQ ID NO: 1 - RNA, 21 nt, Artificial sequence
  Chemically synthesized
cuuacgcugaguacuucgauu

SEQ ID NO: 2 - RNA, 21 nt, Artificial sequence
  Chemically synthesized
uagcgacuaaacacaucaauu

SEQ ID NO: 3 - RNA, 21 nt, Artificial sequence
  Chemically synthesized
agaagaaucuucaguacuauu

(19) United States
(12) Patent Application Publication
(10) Pub. No.: US 2008/0112916 A1
(43) Pub. Date: May 15, 2008

(54) CHEMICALLY MODIFIED POLYCATION POLYMER FOR siRNA DELIVERY

(76) Inventors: Ernst Wagner, Munchen (DE); Julia Klockner, Munchen (DE); Peter Tarcha, Lake Villa, IL (US); Thomas Merdan, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/777,539

(22) Filed: Jul. 13, 2007

Related U.S. Application Data
(63) Continuation of application No. 11/626,174, filed on Jan. 23, 2007.
(60) Provisional application No. 60/761,182, filed on Jan. 23, 2006.
(60) Provisional application No. 60/787,057, filed on Mar. 29, 2006.

(51) Int. Cl. A61K 48/00; C08G 63/48

(52) U.S. Cl. 424/78.3; 514/44; 525/54.2

(57) ABSTRACT

The present invention provides a unique non-viral carrier for nucleic acid delivery in vitro and in vivo, and methods of using thereof.

This application claims the benefit of U.S. Provisional Patent Application No. 60/761,182, filed Jan. 23, 2006, 60/787,057, filed Mar. 29, 2006, and 11/626,174, filed Jan. 23, 2007 the disclosures of each of which are hereby incorporated by reference in their entirety for all purposes.

### BACKGROUND OF THE INVENTION

Non-viral delivery systems for genes have received increasing attention due to the growing implementation of human gene therapy. Cationic lipids formulated into liposomes, and soluble cationic polymers have been demonstrated to readily complex nucleic acid-based drugs and effectively deliver them into cells in vitro. The major barrier on the cellular level is the endosomal membrane, which can be overcome by cationic lipids via a flip-flop mechanism (Xu et al, Biochemistry Vol. 35(18) page 5616 (1996)) or by cationic polymers via the so-called proton-sponge mechanism (Boussif, PNAS Vol. 92(16) page 7297 (1995)). The proton-sponge hypothesis states that during the pH drop in the endosome a polymer may act as a buffer, thus requiring more protons to reach the final pH of approximately 5. This leads to an increased influx of chloride counter ions as well as water, which eventually results in bursting of the vesicle and release of its content into the cytoplasm. Significant in vitro as well as in vivo toxicity is frequently associated with cationic polymers and lipids. On the cellular level the cationic charge leads to membrane damage and vectors may cause necrosis as well as apoptosis. On the in vivo level cationic charge leads to binding to cellular blood components such as erythrocytes and/or non-specific association with serum proteins as well as vessel endothelia. A very rapid clearance by the RES prevents the agent from reaching the intended anatomical sites for intervention. Strategies have been developed to address these undesirable properties, such as charge shielding with proteins and stealth molecules, such as PEG as well as the linkage of active targeting ligands for the intended cells for therapy.

One of the most acceptable and widely used gene delivery polymers is polyethylenimine (PEI). A degradable PEI derivative having many of the desired properties for gene delivery has been described (D. W. Pack, Bioconjugate Chemistry Vol. 14 page 934 (2003)), with a gene delivery activity 16-fold greater than nondegradable 25,000 molecular weight PEI and with low toxicity. It was synthesized by using PEI 800 branched and reacting it with 1,6 hexanedioldiacrylate at a 1 to 1 molar ratio in a Michael fashion. The molecular weight obtained was about 30,000 and based on proton NMR, the structure had numerous biodegradable ester linkages. At pH 5, the half-life for degradation was 30 hours. In a related work partial acetylation of polyethyleneimine with acetic anhydride resulted in up to a 21-fold enhanced gene delivery activity without alteration of the cytotoxicity of the polymers (D. W. Pack Pharmaceutical Research Vol. 21 page 365 (2004)). Other forms of degradable polymer for the therapeutic delivery of polynucleotide-based drugs such as DNA have been obtained using a Michael addition of various amines to various diacrylates (Langer et al., JACS Vol. 123 pages 8155-8156, (2001); JACS Vol. 122, pages 10761-10768, (2000)). In this case the final carrier products had ester bonds and no primary or secondary amines that could participate in an N-acylation reaction; the presence of ester linkages in the polymer backbone improves biodegradability.

Work by Klibanov et al (Pharmaceutical Res. Vol. 22, pages 373-380, (2005)) used two different cross-linking agents, namely, disuccinimidyl suberate (DSS) and ethylene glycol bis[succinimidylsuccinate (EGS) to link both 423-Da and 2 kDa PEI into a higher molecular weight carrier for DNA. The cross-linking agent DSS, after reaction with the amines of PEI produces amide linkages and retains the aliphatic backbone, which originally linked the two amine-reactive groups (active esters) as part of the carrier's structure. This produced amide bonds and retained the aliphatic backbone as part of the structure. The use of EGS with PEI also produced amide linkages, but because of its structure, the carrier had ester linkages as well. Klibanov et al claimed that the material made with EGS was 30 times more efficient in gene delivery than that made with DSS. Klibanov et al suggested “the higher degradability of the EGS-based conjugate facilitates the release of DNA (vector unpacking) and hence enhances the transcriptional availability of DNA”.

Ideally, an optimal non-viral carrier should be a robust polymer with low toxicity, and high gene delivery efficiency. A major advantage of the polymer of the present invention is the ability to deliver a wide range of nucleic acids. While standard polymers such as PEI 25 kDa are efficient in plasmid DNA delivery they are inefficient in delivering siRNAs and no substantial gene expression knockdown can be observed even at higher polymer doses (Kim et al Bioconjugate Chemistry Vol. 17 pages 241-244, 2006). For linear PEI the literature is somewhat contradictory (Hassani et al J Gene Medicine, Vol. 7 pages 198-207, 2005; Urban-Klein et al Gene Therapy Vol 12, 2005), however, data from the literature as well as experiments performed by applicants point out that linear PEI 22 kDa is suitable for knockdown of transiently transfected genes; however, it is not suitable to generate a robust knockdown in stably transfected cell lines. In contrast to these standard reagents the polymer of the present invention is capable of achieving knockdown in transiently as well as stably transfected cell lines and exhibits a high efficiency in plasmid delivery

In summary, there is a need for a nonviral nucleic acid carrier system with the advantages of increased stability as a commercial product, low toxicity, transfection efficiency, the potential ability for elimination by the kidney if the molecular weight is kept low, biodegradability through the amide linkages, and numerous primary and secondary amines through which targeting and stealth ligands may be chemically attached.

### SUMMARY OF THE INVENTION

According to one aspect of the present invention there is provided a compound having the general Formula I:

[[polycation]ₐ-[L]_{b}-[polycation]_{c}]_{d}

wherein:

polycation is a polyethylenimine;

L is an non-ester linker moeity;

a is an integer in the range of about 1 to about 20;

b is an integer in the range of about 1 to about 10;

c is an integer in the range of about 1 to about 20; and

d is an integer in the range of about 1 to about 1000.

Preferably the PEI is oliogethyleneimine (OEI). The polycations of Formula I can optionally have the same polycation recurring or may also have a combination of varying polycations recurring.

“L” or linker of Formula I is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a succinamidyl linker moiety and combinations thereof. The linker moiety is bonded to an amine group contained within the polycation. In a preferred embodiment, the non-ester containing linker moiety is a propionyl unit defined as the chemical group represented by: —CH₂—CHR′—CO—N— where R′ is H or an alkyl group. In a more preferred embodiment, the non-ester linker is a beta-aminopropionylamide linker moiety.

In another embodiment, the compound of Formula I further comprises a biomolecule that is complexed to the compound. The biomolecule may bear one or more an anionic groups and may form an ionic bond with the compound of Formulas I or Ia. Examples of biomolecules bearing one or more anionic groups include nucleic acids (e.g., DNA, single strand RNA, double strand RNA, ribozyme, DNA-RNA bybridizer, siRNA, anitosence DNA and antisence ligo), proteins, peptides, lipids and carbohydrates.

Yet a further embodiment provides a method of transfecting a eukaryotic cell, comprising contacting the cell with such a compound of Formula I and a biomolecule, to thereby deliver the biomolecule to the cell. The method may involve treating a mammal, comprising identifying a mammal in need of gene therapy and administering such a compound to the mammal. In a preferred embodiment, the biomolecule is siRNA, wherein the siRNA is effective to lower expression of a gene of interest.

Another embodiment provides a pharmaceutical composition comprising a compound of Formula I and a biomolecule.

Another embodiment further provides the compound having of general Formula Ia:

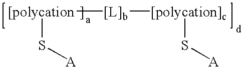

wherein:

polycation is a polyethylenimine;

L a non-ester linker moiety;

S is a spacer or is absent;

A is an agent or is absent;

a is an integer in the range of about 1 to about 20;

b is an integer in the range of about 1 to about 10;

c is an integer in the range of about 1 to about 20; and

d is an integer in the range of about 1 to about 1000.

L or linker of Formula I is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a succinamidyl linker moiety and combinations thereof. The linker moiety is bonded to an amine group contained within the polycation. In a preferred embodiment, the non-ester containing linker moiety is a propionyl unit defined as the chemical group represented by: —CH₂—CHR′—CO—N— where R′ is H or an alkyl group. In a more preferred embodiment, the non-ester linker is a beta-aminopropionylamide linker moiety.

S is a spacer or is absent. The spacer can be, for example, a substituted or unsubstituted, saturated or unsaturated hydrocarbon chain and a substituted or unsubstituted, saturated or unsaturated hydrocarbon chain interrupted by at least one heteroatom such as oxygen, nitrogen and sulfur. Preferably, the hydrocarbon chain comprises 2-20 carbon atoms, more preferably 2-10 carbon atoms and most preferably 2-6 carbon atoms. Suitable spacers may also include but are not limited to polyethylene glycol (PEG).

A is an agent that may facilitate one or more functions in the eukaryotic cell, e.g., receptor recognition, internalization, escape of the biomolecule from cell endosome, nucleus localization, biomolecule release, and system stabilization. The therapeutic agents may include, but not limited to cytotoxic agents, such as paclitaxel, endosomolytic agents, hydrophobic polymers, including but not limited to benzoyl and lauryl groups, targeting moieties, and shielding agents. The shielding agent may include but is not limited to hydrophilic entities, comprising but are not limited to polyethylene glycol (PEG), lactose, sugar, and polyacrylaminde. Targeting ligands include, but are not limited to transferrin, epidermal growth factor, folate, peptides, antibodies or fragments thereof, sugars, and integrin-binding entities such as RGD peptides.

In another embodiment, the compound of Formula Ia further comprises a biomolecule that is complexed to the compound. The biomolecule may bear one or more an anionic groups and may form an ionic bond with the compound of Formula Ia. Examples of biomolecules bearing one or more anionic groups include nucleic acids (e.g., DNA, single strand RNA, double strand RNA, ribozyme, DNA-RNA bybridizer, siRNA, anitosence DNA and antisence ligo), proteins, peptides, lipids and carbohydrates.

In another embodiment, the compounds of Formula Ia further comprise a biomolecule, and an agent (i.e., a shielding, targeting and/or delivery enhancing agent) that is complexed to the compound, optionally including a spacer.

Yet a further embodiment provides a method of transfecting a eukaryotic cell, comprising contacting the cell with such a compound of Formula Ia and a biomolecule, optionally further comprising an agent to thereby deliver the biomolecule to the cell. The method may involve treating a mammal, comprising identifying a mammal in need of gene therapy and administering such a compound to the mammal. In a preferred embodiment, the biomolecule is siRNA, wherein the siRNA is effective to lower expression of a gene of interest.

Another embodiment provides a pharmaceutical composition comprising a compound of Formula Ia and a biomolecule, and may further comprise an agent that is complexed to the polymer.

Yet a further embodiment provides a method of treating a mammal, comprising identifying a mammal in need of gene therapy and administering the compound of Formula I complexes with a biomolecule to a mammal, wherein said biomolecule is siRNA that is effective to lower expression of a gene of interest.

Yet a further embodiment provides a method of treating a mammal, comprising identifying a mammal in need of gene therapy and administering the compound of Formula Ia complexes with a biomolecule to a mammal, wherein said biomolecule is siRNA that is effective to lower expression of a gene of interest.

### BRIEF DESCRIPTION OF THE FIGURES

FIG. 1a-1b. Proposed mechanism of polycation modification.

FIG. 2. Infrared spectra for OEI-HD product.

FIG. 3a-3b. Structural elements and IR for PEI-800 modified with suberic acid chloride.

FIG. 4. siRNA delivery HUH7/EGPLuc cells using OEI-HD-1.

FIG. 5. siRNA knockdown with OEI-HD-1 in different media.

FIG. 6a-6b. siRNA knockdown with OEI-HD-1 in media containing serum.

FIG. 7. Failed siRNA knockdown with OEI-SUB-1.

FIG. 8a-8b. Beta-aminopropionylamide linker examples.

FIG. 9a-9b. Effect of siRNA on knockdown of luciferase activity in stably transfected Neuro2A-EGFPLuc cells using OEI-HD, different amount of siRNA per well and various w/w ratios (a). Comparison of knockdown efficiency by linear PEI 22 kDa (PEI22), branched PEI 25 kDa (PEI25), and OEI-HD using 0.25 μg siRNA in the formulations at indicated w/w ratios (b). Formulations were prepared in HBG, siRNA delivery was performed in medium containing 10% serum without medium change in 96-well plates with 5,000 cells/well using Luc siRNA (GL3) and Mut siRNA (siCONTROL). Luciferase expression was measured in triplicates two days following siRNA delivery.

FIG. 10. siRNA delivery to Neuro2A-EGFPLuc cells using different percentage of transferrin (Tf) conjugate as shielding and targeting reagent. Formulations were prepared in HBG, siRNA delivery was performed using 0.50 μg siRNA and OEI-HD/siRNA ratio of 2/1 in medium containing 10% serum in 96-well plates with 5,000 cells/well without medium change. Luciferase expression was measured in triplicates two days following siRNA delivery.

FIG. 11. Cytotoxic activity of RAN siRNA on Neuro2A cells using OEI-HD derivative, various amount of siRNA and polymer/siRNA ratios of 2/1 (a) or 0.6/1 (b) for siRNA delivery. Transfection was performed in medium containing 10% serum in 96-well plates with 4,000 cells/well without medium change. As a control, non-targeted control Mut siRNA (siCONTROL) was used. Cell viability was determined 3 days following application using MTT assay.

FIGS. 12a, 12b and 12c. (a) Quantitative analysis of Ran protein expression in Neuro2A cells following RAN specific siRNA application in comparison to unspecific Mut siRNA (siCONTROL) and non-treated cells; (b) RAN siRNA expression using western blot analysis; (c) Expression of α-tubulin as a housekeeping gene that was used for normalization of protein content in each sample.

FIG. 13a-13b. (a) Quantitative analysis of Ran-protein expression in A/J-mice bearing Neuro2A tumors by western blot following application of Tf-shielded RAN siRNA polyplexes in comparison to unspecific siRNA (Mut siRNA) polyplexes and non-treated animals. (b) Protein expression levels of each sample were normalized for the expression of α-tubulin set as 100%. Samples were assessed two days after the last of three systemic applications. Data shown are mean ±SD. All data sets collected have a group size of ten animals; data points marked with asterisks are statistically significant (*p<0.05, **p<0.01; Mann-Wilcox-U-test).

FIG. 14a-14b. Apoptosis in Neuro2A tumors (a) and liver (b) following application of Tf-shielded RAN siRNA polyplexes compared to non-specific siRNA (Mut siRNA) or non-treated animals by using TUNEL staining of 5 μm tissue sections. The apoptotic fraction was derived by counting the TUNEL-positive fractions (green) of 100 cells from up to 20 random fields in each tumor section. DAPI was used for nuclear counterstaining (blue). Upper part of the figure shows selected examples of staining demonstrating the average findings. Lower part shows the statistical evaluation of all fractions analyzed. Values are means from five animals per group ±SD.

FIG. 15a-15b. Biological effects of Tf-shielded OEI-HD/siRNA formulations compared to unspecific siRNA complexes and non-treated animals. Whole blood samples were obtained from A/J mice (5 animals per group) 48h following the last of three injections and measured for blood counts (a) and liver enzymes (b) Data shown are mean levels ±SD.

### DETAILED DESCRIPTION OF THE INVENTION

The present invention relates to a unique nonviral carrier for biomolecule delivery, wherein the carrier is a polymer having polycations chemically linked by propionylamide units. The present invention further relates to compounds of Formulas I and Ia described below, methods of preparing said compounds, as well as method of using the compounds of Formulas I and Ia.

### I. Compound of Formula I

An embodiment provides polycations chemically linked by proprionylamide units as described in Formula I:

[[polycation]ₐ-[L]_{b}-[polycation]_{c}]_{d}

In Formula I, the polycation is defined as a molecule capable of obtaining more than two cationic charge when placed into aqueous solution. For example, in certain embodiments the polycation of Formula I may include, but are not limited to, polyethylenimine 400 Da-750 kDa, dendrimer structures (e.g. polypropyleneimine dendrimers or PAMAM dendrimers with different structures and molecular weight), spermine, spermidine, triethylenetetramine, tetraethylenpentamine, and pentaethylenhexamine. Preferably the polycation of the Formula I is a poly-ethyleneimine (PEI) and most preferably the polycation of Formula I is oliogethyleneimine (OEI). The polycations of Formula I can optionally have the same polycation recurring or may also have a combination of varying polycations recurring.

“L” or linker of Formula I is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a succinamidyl linker moiety and combinations thereof. The linker moiety is bonded to an amine group contained within the polycation. In a preferred embodiment, the non-ester containing linker moiety is a propionyl unit defined as the chemical group represented by: —CH₂—CHR′—CO—N— where R′ is H or an alkyl group. In a more preferred embodiment, the non-ester linker is a beta-aminopropionylamide linker moiety.

The polycation may contain recurring units of the same polycation or a combination of varying polycations, in which a and c are integers in the range of about 1 to about 20. Moreover, L can be recurring, also with either the same linker moiety or with a combination of varying linker moieties and therefore b is an integer in the range of about 1 to about 10. The entire compound of Formula I one can also be recurring and d is an integer in the range of about 1 to about 100 and is preferably in the range of about 30.

### II. Compound of Formula Ia

A further embodiment provides polycations chemically linked by proprionylamide units as described in Formula Ia:

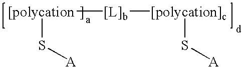

wherein:

“S” is a spacer or is absent. The spacer can be, for example, a substituted or unsubstituted, saturated or unsaturated hydrocarbon chain and a substituted or unsubstituted, saturated or unsaturated hydrocarbon chain interrupted by at least one heteroatom such as oxygen, nitrogen and sulfur. Preferably, the hydrocarbond chain comprises 2-20 carbon atoms, more preferably 2-10 carbon atoms and most preferably 2-6 carbon atoms. Suitable spacers may also include but are not limited to polyethylene glycol (PEG).

In a further embodiment of compounds of Formula Ia the may also include an agent (“A”). “A” is an agent or is absent. Preferably, “A” is an agent that may facilitate one or more functions in the eukaryotic cell, e.g., receptor recognition, internalization, escape of the biomolecule from cell endosome, nucleus localization, biomolecule release, and system stabilization. The therapeutic agents may include, but not limited to cytotoxic agents, such as paclitaxel, endosomolytic agents, hydrophobic polymers, including but not limited to benzoyl and lauryl groups, targeting moieties, and shielding agents. The shielding agent may include but is not limited to hydrophilic entities, comprising but are not limited to polyethylene glycol (PEG), lactose, sugar, and polyacrylaminde. Targeting ligands include, but are not limited to transferrin, epidermal growth factor, folate, antibodies or fragments thereof, peptides, sugars, and integrin-binding entities such as RGD peptides.

It is understood that at least on of S and A must be present in Formula Ia.

The polycation may contain recurring units of the same polycation or a combination of varying polycations, in which a and c are integers in the range of about 1 to about 20. Moreover, L can be recurring, also with either the same linker moiety or with a combination of varying linker moieties and therefore b is an integer in the range of about 1 to about 10. The entire compound of Formula Ia one can also be recurring and d is an integer in the range of about 1 to about 100 and is preferably in the range of about 30.

The molecular weight of the compound of Formula I may range from about 800 Daltons to about 1,000,000 Daltons, preferably in the range of about 20,000 Daltons to about 200,000 Daltons, and most preferably in the range of about 20,000 Daltons to about 30,000.

The molecular weight of the compound of Formula Ia may range from about 800 Daltons to about 1,000,000 Daltons, preferably in the range of about 20,000 Daltons to about 200,000 Daltons, and most preferably in the range of about 20,000 Daltons to about 30,000.

The molar ration of polycation to L is 20-50. While the molar ration of free amines on the polycation to agents can vary depending on agent and may be from about 1000 and 2.

### III. The Compounds of Formula I or Ia in Complex with Biomolecules

The compound of Formula I may form complexes with biomolecules and thus are useful as carriers for the delivery of biomolecules to cells. Examples of biomolecules that form complexes with the compound of the Formula I include nucleic acids, proteins, peptides, lipids, and carbohydrates. Examples of nucleic acids include DNA, single strand RNA, double strand RNA, ribozyme, DNA-RNA hybridizer, and antisense DNA, e.g., antisense oligo. A preferred nucleic acid is siRNA. Cationic lipopolymers that comprise a biomolecule that is complexed to the polymer may be formed by intermixing the cationic lipopolymers and biomolecules in a mutual solvent, more preferably by the methods described in the examples below.

### IV. The Compounds of Formula I or Ia in Complex with Biomolecules and Optionally with Agents

The polymer of the present invention can also form an ionic complex or covalent bond with specific therapeutic agents, including but not limited to cytotoxic agents, such as paclitaxel, endosomolytic agents, hydrophobic polymers and other targeting moieties.

The OEI-HD carrier can be modified with shielding ligands, which will reduce the occurrence of unwanted non-specific interactions after in vivo administration, and therefore improve circulation lifetime after administration. Shielding ligands comprise hydrophilic entities, comprising but are not limited to polyethylene glycol (PEG), lactose, sugar, and polyacrylaminde. Additionally, to improve uptake into the tissue of interest, the carrier, which can be OEI-HD or shielded OEI-HD, can have targeting ligands conjugated to the same. Examples of targeting ligands comprise but are not limited to transferrin, epidermal growth factor, folate, antibodies or fragments thereof, sugars, and integrin-binding entities such as RGD peptides.

### V. The Preparation of the Compounds of Formula I and Ia

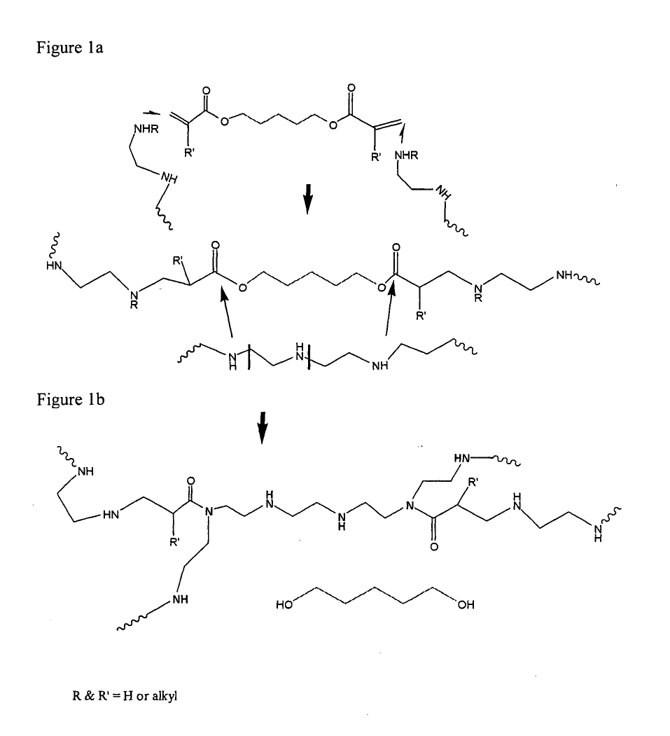

The cross-linking of the polycation, preferably a poly-ethyleneimine (PEI), occurs by the Michael addition of a fraction of the polymer's amines to vinylic groups of cross-linking groups and from N-acylation of pendant ester groups. Cross-linking groups, can be acrylate or methacrylate ester monomers. It is understood that acrylate and methacrylate ester monomers comprise several groups including but not limited to diacrylate, dialkylacrylate, dimethacrylate, diacrylate ester monomers. Acylation is defined as the introduction of an acyl group into the molecule of an organic compound having hydroxyl (O-acylation) or amino (N-acylation) groups. Additional N-acylation of the polymer can be achieved by the reaction of esters, such as ethyl acetate and anhydrides with the polymer. The chemical modifications may occur at both the primary and secondary amines of the polymer structure, thus reducing the net number of ionizable groups. If multifunctional reactants are used for the modification, an increase in the average molecular weight of the polymer occurs. These mechanisms and chemical units introduced are illustrated in FIG. 1.

The resulting polymer has application as a nonviral synthetic carrier for a variety of entities with opposite charge, including but not limited to nucleic acid and therapeutic peptides. A major advantage of the polymer of the present invention is the ability to deliver a wide range of nucleic acids. While standard polymers such as PEI 25 kDa are efficient in plasmid DNA delivery, they are inefficient in delivering siRNAs and no substantial gene expression knockdown can be observed even at higher polymer doses (Kim et al Bioconjugate Chemistry Vol. 17, pages 241-244, 2006). For linear PEI the literature is somewhat contradictory (Hassani et al J Gene Medicine Vol 7, pages 198-207, 2005; Urban-Klein et al Gene Therapy Vol 12 2005), however data from the literature as well as experiments by applications point out that linear PEI 22 kDa is suitable for knockdown of transiently transfected genes, however it is not suitable to generate a robust knockdown in stably transfected cell lines. In contrast to these standard reagents the polymer of the present invention is capable of achieving knockdown in transiently as well as stably transfected cell lines and exhibits a high efficiency in plasmid delivery. Therefore double stranded RNA molecules such as small interfering RNAs (siRNA) are uniquely suitable for delivery with the polymer carrier of the present invention. The siRNA delivery can be for therapeutic purposes or for target validation, i.e. identification of potential targets for novel therapeutic purposes.

The polymer carrier of this invention can be prepared from various oligoamines including but not limited to dendrimer structures (e.g. polypropyleneimine dendrimers or PAMAM dendrimers with different structures and molecular weights), spermine, spermidine, triethylentetramine, tetraethylenpentamine, and pentaethylenhexamine or from a base material consisting of branched polyethylene imine, with a 400 to 25,000 MW range. The branched polyethylene imine is chemically modified or cross-linked with mono- bi- or multi functional agents. The polyethylene imine contains a plethora of primary, secondary and tertiary amines and those amines make up approximately 30% of the polymer mass. Both the primary and secondary amines are available for reaction with the cross-linking agent. A crosslinking agent is defined as a molecule that has at least 2 reactive groups and is used to chemically link at least 2 polymer molecules. Agents that can be used as a cross-linking agent include but are not limited to ethylene glycol diacrylate, ethylene glycol dimethacrylate, 1,6 hexanediol diacrylate, polyethylene glycol 600 diacrylate and other di- or multiacrylate or di- or multi-methcrylate molecules. The preferred agent used in this invention is a diacrylate, namely 1,6 hexanediol diacrylate abbreviated as HD. HD has four possible reactive sites, namely 2 vinylic groups and 2 ester groups. Since the polyethyleneimine is of low molecular weight we commonly refer to it as oliogethyleneimine, abbreviated as OEI. When the OEI is combined with the HD and heated for several days, the reactive amines add across the two sites of unsaturation in a Michael fashion.

Normally the ratio of HD to OEI used is one-to-one, on a molar basis. When the ratio is more than one-to-one, then the numerical designation reflects the molar ratio, for example OEI-HD-1 means that a HD to OEI ratio is one-to-one, OEI-HD-5 means that a HD to OEI ratio is five-to-one. When the reaction is complete, proton NMR shows that all of the vinylic groups have been consumed and the resulting product is usually soluble in water. So crosslinking of the OEI chains has not occurred to an extent that a swellable hydrogel has formed. Size exclusion chromatography of a typical sample gave a chromatogram which allowed the calculation of the number and weight average molecular weight and hence the polydispersity (weight average molecular weight divided by number average molecular weight or Mw/Mn). These values were 3000, 16,000 and 5.3, respectively. A polydispersity value of 1 indicates a monodisperse molecular weight. Value of 2-3 are somewhat narrow whereas 4 and above indicate a broader distribution.

This polymer was designed to be less toxic than higher molecular weight polyethyleneimine, (PEI) 25,000, the levels of which are unacceptable for in vivo use in gene transfection or siRNA delivery. Low molecular weight PEI's, like OEI 800, used as a starting material in this invention, is relatively non toxic, but not very effective at delivering nucleic acids across a cell membrane. The product from the reaction described above is both relatively non toxic to cells and very effective at delivering siRNA and DNA across cell membranes. It also releases them into the cytoplasm, so they can perform a biological effect in the cell. An additional feature of the idealized structure shown above is that it should be biodegradable by means of the ester linkages, which can be part of the structure. In theory, hexanediol could be obtained upon ester hydrolysis as a by-product as well as OEI 800 containing 1-alkylamino-propanoic acid end groups.

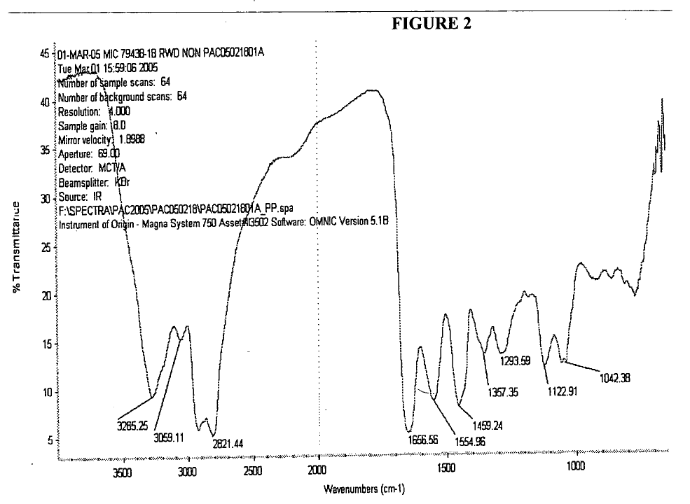

Infrared spectra were run on such carriers after synthesis and isolation, but we were surprised to find that very little ester was present, and a strong amide peak was dominant. Carboxylic acid was not indicated in the infrared spectrum either, which seemed to rule out premature ester hydrolysis (FIG. 2). Proton NMR of the product after dialysis against distilled water indicated an absence of aliphatic methylene groups which should be part of the HD linkages. A reaction that can explain these results is the acylation of the ester bonds by residual amines on the OEI, which should be in abundance based on the molar ratio of starting materials. Such acylation would also provide a by-product of 1,6 hexanediol, which is water soluble and removable by dialysis. Further evidence of ester acylation by amines on the OEI was provided by washing of the product with ethyl acetate, a non-solvent for the polymer. In this case analysis indicated that a limited amount of N-acylation of ethyacetate occurred.

A similar polycation carrier can be obtained by a two-step process. In this alternative two-step process, the first step consist in the core polycation being modified with a crosslinking agent, and the second step consist in a further modification by addition of a similar or different type of polycation, for example, spermine and pentaethylenhexamine.

### EXAMPLES

The present invention will be further clarified by the following examples, which are only intended to illustrate the present invention and are not intended to limit the scope of the present invention.

### Example 1

### Synthesis of the Carrier OEI-HD-1

5.0 g (0.0063 moles) of polyethylene imine (weight average molecular weight 800) were dissolved in 7.5 ml of DMSO. In a separate container, 3.3 ml of DMSO and 1.4 ml=1.4 g (0.0063 moles) 1,6 hexanediol diacrylate were added. Both solutions were mixed. In a 50 ml round bottom flask immersed in oil bath thermostated at 60 degrees C. and fitted with a magnetic stir bar. The flask was loosely stoppered and allowed to react for 4 days. Then the reaction solution was added dropwise to 200 ml of a rapidly stirred solution of ethyl acetate whereby a viscous material formed on the bottom and sides of the flask. The solvent was decanted off and a fresh 200 ml aliquot of ethyl acetate was added and the materials mixed. This was decanted again and an additional 100 ml aliquot was added, mixed and decanted leaving behind the viscous material. The material was transferred to a boat made from aluminum foil and it was placed in a vacuum oven at room temperature overnight. This evacuation process fails to remove all of the ethyl acetate because of the low surface area and high viscosity of the material and required further purification.

### Example 2

### Purification of OEI-HD-1 by Dialysis

Weighted out 0.80 g of OEI-HD-1 and added it to a scintillation vial followed by 10 ml of Dulbucceos PBS buffer. It dissolved after a short time with shaking. Preconditioned about I linear foot of Spectrum 3500 cut-off dialysis membrane (0.4 ml/cm of length capacity) by boiling it in a beaker of distilled water for about 10 minutes. Then a knot was tied in one end of the dialysis tubing and the OEI-HD-1 solution was added and sealed by tying a knot in the other end. The tube was placed in approximately 3 gallons of distilled water and the water was stirred gently for 4 days. After that the material was removed from the tubing and freeze dried yielding about 30 percent of the polymer mass that was added to the tubing. Proton and Carbon 13 NMR were run on this product.

### Example 3

### Precipitation of OEI-HD-1 into Dioxane

Example 1 is repeated but instead of using ethyl acetate for washing, dioxane was substituted. The use of dioxane avoids the possibility of acetylation of free amines by the ethyl acetate ester.

### Example 4

### Synthesis of OEI-HD-5

Dissolved 5.0 g (0.0063 moles) of polyethylene imine (weight average molecular weight 800) in 7.5 ml of DMSO in a 125 ml glass bottle. To a separate container, added 17 ml of DMSO and 7.0 ml=7.0 g (0.0315 moles) 1,6 hexanediol diacrylate and mixed. Combined both solutions in the 125 ml bottle, loosely capped it and placed it in a oven thermostated at 60 degrees C. After about 30 minutes into the reaction, a gel was seen to form. The reaction was allowed to continue for a total of 3 days. Then the DMSO was decanted from the gel and the gel was broken up into chunks with a spatula. A small chunk was placed in a 20 ml scintillation vial with about 10 ml of water whereupon most of the gel appeared to dissolve.

### Example 5

### Synthesis of OEI-HD-10

Dissolved 5.0 g (0.0063 moles) of polyethylene imine (weight average molecular weight 800) in 7.5 ml of DMSO in a 125 ml glass bottle. To a separate container, added 34 ml of DMSO and 14 ml=14 g (0.0630 moles) 1,6 hexanediol diacrylate and mixed. Combined both solutions in the 125 ml bottle, loosely capped it and placed it in a oven thermostated at 60 degrees C. After about 30 minutes into the reaction, a gel was seen to form. The reaction was allowed to continue for a total of 3 days. Then the DMSO was decanted from the gel and the gel was broken up into chunks with a spatula. A small chunk was placed in a 20 ml scintillation vial with about 10 ml of water and shaken for 2 days. None of the material dissolved.

### Example 6

### Synthesis of OEI-Sub-1

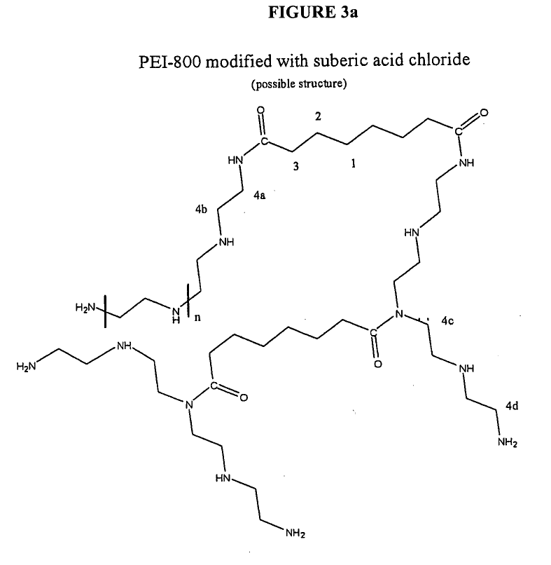

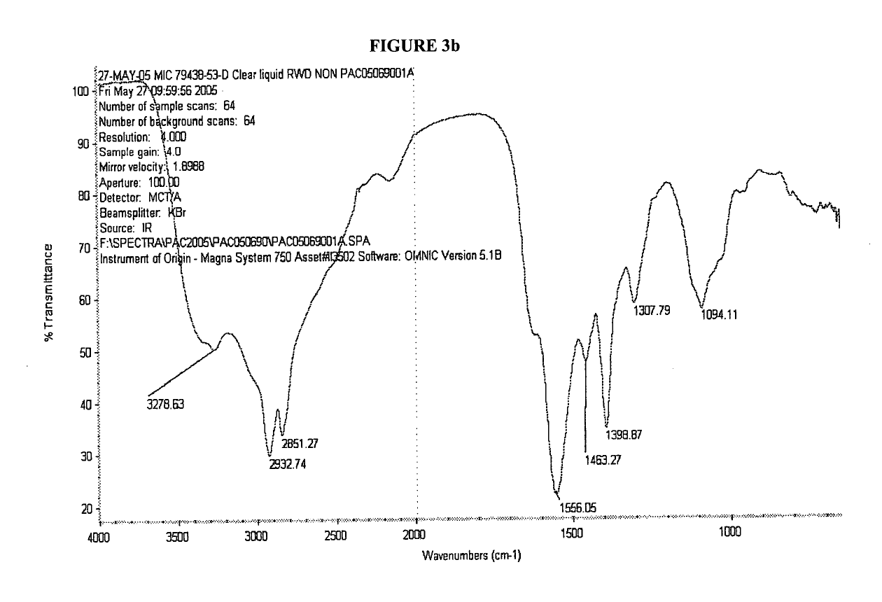

Weighed out 1.0 g of PEI-800, (0.0013 moles) and added 5 ml of DMSO that had been dried by standing over magnesium chloride. The PEI was not completely soluble, but formed a cloudy suspension. Pipetted 0.22 ml (0.26 g, density 1.172, 0.0013 moles) of suberoyl chloride into 5 ml of dry DMSO. All glassware was flamed to remove moisture. Added the suberoyl solution dropwise to the PEI solution/suspension at room temperature with shaking by hand. An insoluble gel-like material formed immediately. The gel was washed 1× with excess fresh dry DMSO followed by 2× washes with excess dioxane. The residual solvent was decanted off and the gel was left under house vacuum at room temperature for 19 hours. The material had a noticeable stench even after vacuum drying. A sample was submitted for IR (microscope) and proton NMR in D₂O. The proton NMR indicated the presence of residual DMSO and dioxane, in addition to the expected water. Removed 50 mg for a file sample and completely dissolved the remainder of the sample in about 10 ml of distilled water. The sample was placed in a dialysis tube (3500 molecular weight cut-off) and dialyzed against about 12 liters of distilled water with gentle stirring with a magnetic stir bar for 5 days. The sample was divided into two vials and freeze dried. Approximately 100 mg of sample was recovered after freeze drying. Correcting for the 50 mg that was retained as an impure file sample, about 90% of the material was lost, most likely by passing through the dialysis membrane. The proposed structure of PEI-800-Sub-1 is shown in FIG. 3a and the IR of the product is graphically represented in FIG. 3b.

### Example 7

### SiRNA Knockdown with OEI-HD-1

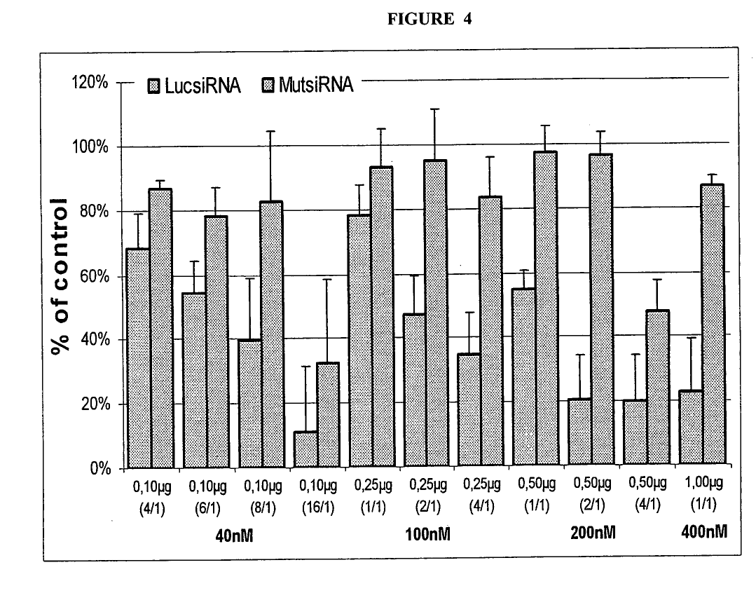

The results of siRNA delivery on HUH7/EGFPLuc cells using OEI-HD-1 are summarized in FIG. 4. Transfections were performed in 96-well-plates using 5,000 cells/well in serum-free medium (OptiMEM). OEI-HD-1/siRNA formulations were prepared in 20 μl HBS (20 mM HEPES, 150 mM NaCl) and added to 80 μl of serum-free medium (100 μl total volume). Four hours following delivery, transfection medium was replaced by growth medium and two days later luciferase activity was measured. Using 0.10 μg siRNA (40 nM) and the C/P ratio 8/1 (OEI-HD-1/siRNA: w/w) up to 50% knockdown of luciferase activity was achieve compared to transfection using unspecific MutsiRNA. With the purpose of clarification, C/P ratio means the carrier to plasmid weight ratio, which for the purpose of the present invention can be DNA or siRNA. The MutsiRNA is used as a control and is a good measure of the toxicity of the carrier. So if a reduced signal is seen with the MutsiRNA, which should have no biological activity, the knockdown seen with the specific siRNA at the same concentration should be corrected for the toxic effect of the carrier on the cells.

Using 0.25 μg siRNA (100 nM) and the C/P ratio 4/1 up to 60% knockdown of luciferase activity was achieved compared to transfection using unspecific MutsiRNA. For 0.50 μg siRNA (200 nM) up to 80% knockdown was observed. The use of higher concentration of siRNA (up to 1.00 μg siRNA (400 nM) did not lead to any further reduction of luciferase activity. Thus for the delivery in HUH7/EGFPLuc using OEI-HD-1 (complexes in HBS, transfection in serum-free medium) the maximal knockdown of luciferase expression can be achieved by 200 nM (0.50 μg siRNA per 5,000 cells) and the C/P ratio 2/1.

### Example 8

### SiRNA Knockdown with OEI-HD-1 in Different Buffer Media

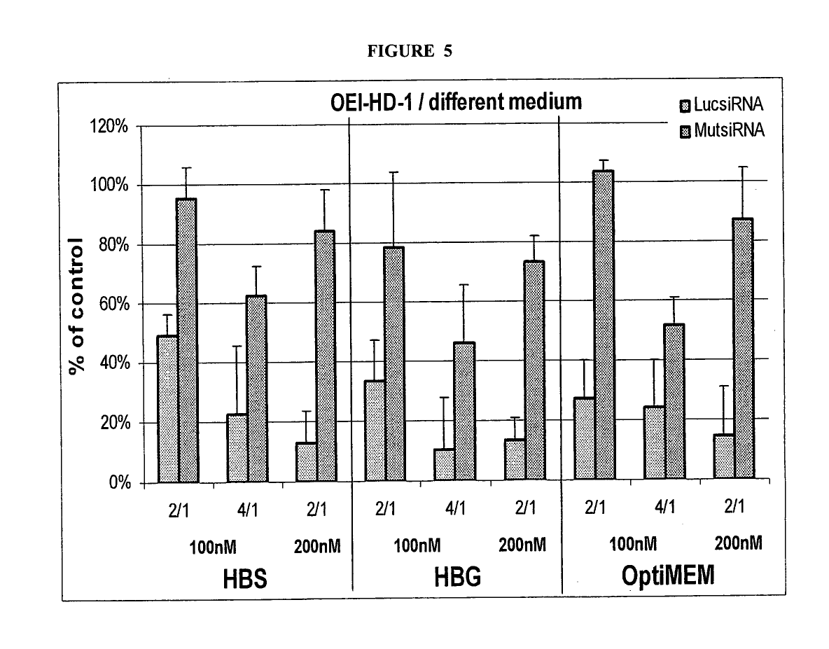

The ability of OEI-HD-1 to knockdown the luciferase expression was tested in different serum-free complexation media (HBS: 20 mM HEPES, 150 mM NaCl; HBG: 20 mM HEPES, 5% glucose; OptiMEM: salt reduced serum-free medium, Gibco). Independent of the complexation medium used, OEI-HD-1/siRNA formulations were able to knockdown of luciferase activity for up to 80% without significant differences between the complexation media. Formulations in OptiMEM were high efficient at 100 nM (C/P: 2/1). This may be caused by the faster aggregation of OEI-HD-1/siRNA particles. Results are shown in FIG. 5.

### Example 9

### SiRNA Knockdown with OEI-HD-1 in Media Containing Serum

siRNA delivery in HUH7/EGFPLuc was performed using OEI-HD-1 in the presence of serum (10% FCS). OEI-HD-1/siRNA formulations were prepared in HBS (20 μl) and complexes were added to 80 μl of serum containing medium on the cells. Two different approaches were performed; first, medium was changed four hours following siRNA delivery as usually and second medium wasn't changed for two days. Following medium change, maximal knockdown of luciferase expression was achieved using 200 nM siRNA and the C/P ratio 6/1, which was in contrast to the optimal transfer conditions achieved in serum-free medium: 200 nM siRNA, C/P ratio 2/1.

Without medium change, the optimal transfer conditions for OEI-HD-1/siRNA delivery were the same as in the absence of serum (200 nM siRNA, C/P ratio 2/1). Furthermore, without medium change, OEI-HD-1 was much more toxic and at the C/P ratio 6/1 and 200 nM the cells died. Interestingly, w/o medium change, already 100 nM siRNA and the C/P ratio 4/1 were high efficient for expression knockdown of the luciferase gene.

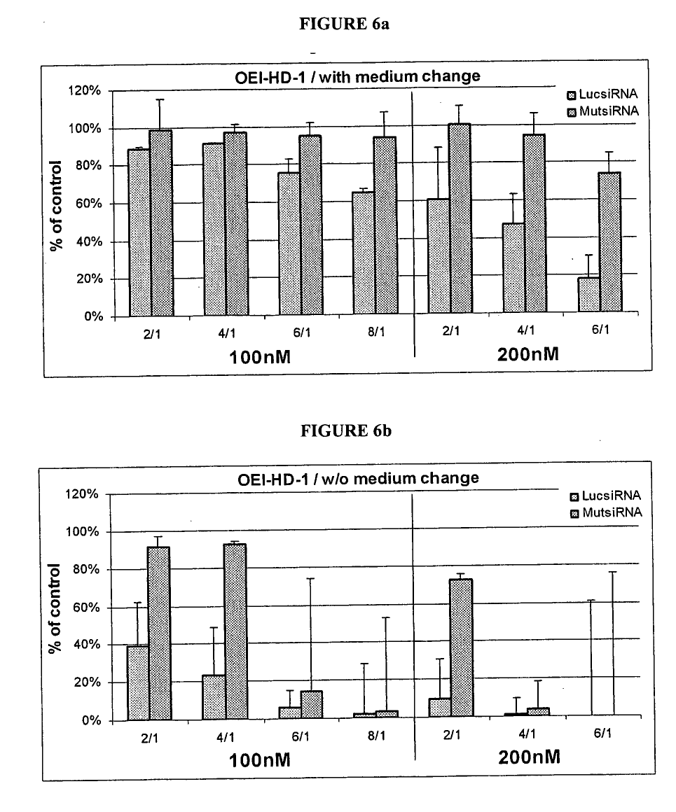

In summary, OEI-HD-1 vehicle was also efficient for siRNA delivery in the presence of serum. Similar to results in serum-free medium up to 80% knockdown of luciferase expression was observed also in the presence of 10% FCS. Furthermore, without medium change, already lower siRNA concentration was sufficient to achieve maximal effect. Results are summarized in FIGS. 6a and 6b.

### Example 10

### Failed SiRNA Knockdown with OEI-Sub-1

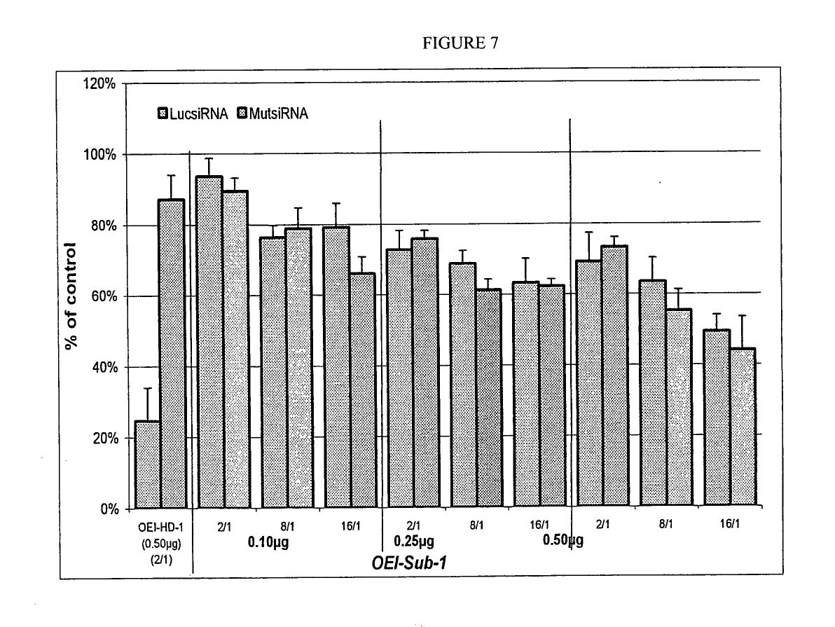

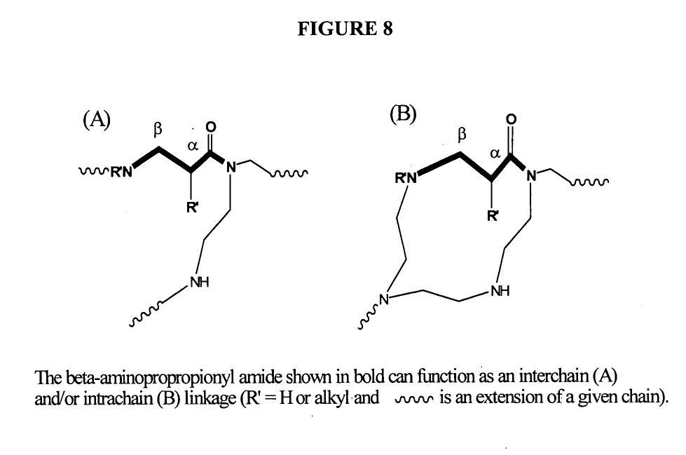

OEI-Sub-1 synthesized using OEI (800 Da) and suberoyl acid (C₆H₁₂O₂Cl₂) as a cross linker (as described in Example 6) was tested for siRNA delivery on HUH7/EGFPLuc cells using different polymer/siRNA ratios. Transfection was performed in 96-well plates and 5,000 cells/well in triplicates using LucsiRNA (GL3) and MutsiRNA (IX) (Dharmacon). OEI-complexes were prepared in HBS and siRNA delivery was performed in serum-free medium for 4 hours. Transfection medium was then replaced by growth medium and luciferase expression was measured two days following siRNA delivery. The amount of siRNA was varied from 0.1 to 0.50 μg per 5,000 cells and OEI-Sub-1/siRNA ratio was varied as well; however, no silencing of luciferase gene expression was observed as shown in FIG. 7.

### Example 11

### Coupling of K5 Peptide Mimetic to OEI-HD-1

OEI-HD (2.8 mg) is dissolved in 1 mL of reaction buffer containing 150 mM sodium chloride and 20 mM 4-(2-hydroxyethyl)-1-piperazine ethane sulfonic acid (HEPES), pH 7.5. N-succinimidyl 3-(2-pyridyldithio)propionate (SPDP) (80 □g) is added to this solution in 80 □L of 100% ethanol while stirring. The reaction is allowed to continue for 90 min. SPDP-activated OEI-HD is then purified by gel filtration using Sephadex G-25. In the second step, a 3-fold molar excess of K5 peptide bearing a cysteine on the N-terminus is added in the same buffer. The reaction is allowed to proceed for 12 h at room temperature and then purified by gel filtration using Sephadex G-25.

### Example 12

### Coupling of RGDC (Cysteine Terminated) Peptide to OEI-HD-1

OEI-HD (2.8 mg) is dissolved in 1 mL of reaction buffer containing 150 mM sodium chloride and 20 mM 4-(2-hydroxyethyl)-1-piperazine ethane sulfonic acid (HEPES) pH 7.5. N-succinimidyl 3-(2-pyridyldithio)propionate (SPDP) (80 μg) is added to this solution in 80 μL of 100% ethanol while stirring. The reaction is allowed to continue for 90 min. SPDP-activated OEI is then purified by gel filtration using Sephadex G-25. In the second step, a 3-fold molar excess of RGDC peptide is added in the same buffer. The reaction is allowed to proceed for 12 h at room temperature and purified by gel filtration using Sephadex G-25.

### Example 13

### Coupling of Antibody Fragment (Fab′)

Using a procedure derived from Merdan et al. Bioconjugate Chemistry Vol. 14, pages 989 (2003), OEI-HD (2.8 mg) is dissolved in 1 mL of reaction buffer containing 150 mM sodium chloride and 20 mM 4-(2-hydroxyethyl)-1-piperazine ethane sulfonic acid (HEPES) pH 7.5. N-succinimidyl 3-(2-pyridyldithio)propionate (SPDP) (80 μg) is added to this solution in 80 μL of 100% ethanol while stirring. The reaction is allowed to continue for 90 min. SPDP-activated OEI is then purified by gel filtration using Sephadex G-25.

In the second step, a 1.4-fold molar excess of freshly reduced Fab′ is added in the same buffer. The reaction is allowed to proceed for 12 h at room temperature and purification is performed by gradient ion exchange chromatography using 0.9% NaCl, 10 mM HEPES pH 7.4 as buffer A and 3 M NaCl, 10 mM HEPES pH 7.4 as buffer B and MacroPrep HighS (from Amersham Pharmacia).

### Example 14

### Coupling of Polyethylenglycol to OEI-HD Via HMDI Activation

PEG monomethyl ether (0.5-20 kDa) is dissolved in anhydrous chloroform (200 g/L) and activated with a 10-fold excess of hexamethylene diisocyanate, HMDI at 60° C. for 24 h. Unreacted HMDI is carefully removed by repetitive extraction with light petrol. Subsequently the reaction of the isocyanate-terminated PEG with the amino groups of OEI-HD is carried out in anhydrous chloroform at 60° C. for 24 h. The degree of PEGylation can be adjusted by varying the ratio of activated PEG to OEI-HD. The reaction solution is precipitated in diethyl ether or other suitable non-solvent and the product is dried in vacuo.

### Example 15

### Coupling of Polyethyleneglycol to OEI-HD Via PEG-NHS

PEG-N-hydroxy succinimidyl esters of the desired molecular weight are dissolved in DMSO and added to an aqueous solution of OEI-HD-1. The reaction is stirred for 24 hours and the pegylated polymer is isolated by e.g. size exclusion chromatography.

### Example 16

### Coupling of a Cyclic RGD Peptide Via PEG-Spacer to Form a PEG-Shielded Targetable Carrier

RGD is coupled to polyethylene glycol following a procedure published in Nucleic Acids Research, Vol 32 page 149, 2004. A chemical conjugate of polyethylene glycol with RGD (H-ACRGDMFGCA-OH) is synthesized first. This is done by oxidizing the two cysteine residues forming a cyclic 10-mer RGD peptide with a disulfide bridge. Then 60 mg of the cyclic peptide is dissolved in 600 μl dimethyl sulfoxide (DMSO). Triethylamine (TEA) 8.54 μl, pre-dissolved in 20 μl of tetrahydrofuran, is added to the peptide under nitrogen. After stirring for 1 min, a solution of activated PEG, namely, NHS-PEG-VS (212 mg in THF: DMSO; 300 μl: 100 μl) is added in one portion to react the n-hydroxysuccinimide (NHS) group on the PEG with the amino terminus of the peptide. The reaction mixture is stirred at room temperature for 4 h and quenched with trifluoroacetic acid (TFA) at equivalence to the TEA. The RGD-PEG-VS is purified by dialysis against distilled water followed by lyophilization. In the second step, 100 mg (21.7 μmol) of the purified RGD-PEG-VS intermediate is dissolved in 1 ml of anhydrous DMSO. To this solution, six equivalents of TEA dissolved in 0.5 ml THF is added and mixed. An aliquot of 9.4 mg (218 μmol in terms of amines) of OEI-HD dissolved in dimethylformamide (0.5 ml) is added to this solution and stirred at room temperature for 12 h to cause Michael addition of amines on the OEI with the vinyl sulfone (VS) on the PEG. The product is purified as the TFA salt by HPLC.

### Example 17

### OEI-HD Prodrug

Paclitaxel is coupled to one end of a polyethylene glycol molecule as described (Materials Research Innovations Vol. 9 pages 13-14, 2005). The other hydroxyl end of the PEG chain is reacted with trichloro-s-triazine to make an activated PEG end group. The activated PEG is combined with OEI-HD in water at pH 9.0 to allow coupling of the PEG-paclitaxel to the OEI-HD carrier. This OEI-HD-PEG-paclitaxel moiety is combined with other OEI-HD polymers having targeting ligands as well as specific siRNA against the cells of interest to form a polyplex. This polyplex has properties of delivering agents that interfere with cellular transcription (siRNA) as well as delivering small cytotoxic agents i.e, paclitaxel.

### Example 18

### In Vivo Use of Chemically Modified Polycation for RAN-siRNA Delivery

Transfection Reagents and Chemicals

The plasmid pEGFPLuc (Clontech Laboratories, Heidelberg, Germany) containing a CMV promoter driven fusion of the genes encoding for enhanced green fluorescent protein and luciferase was used for generation of stably transfected cells. Lipofectamine 2000 (LF2000) was obtained from Invitrogen (Karlsruhe, Germany). Linear PEI with an average molecular weight of 22 kDa (PEI22) was synthesized by acid-catalysed deprotection of poly(2-ethyl-2-oxazoline) (50 kDa, Aldrich) in analogous form as commercially available from Polyplus (Straβbourg, France). Branched PEI with an average molecular weight of 25 kDa (PEI25) was obtained from Sigma-Aldrich (Munich, Germany). PEIs were used as a 1-10 mg/ml stock solution neutralized with HCl. Transferrin was obtained from Biotest (Dreieich, Germany). NHS-PEG-MAL with a molecular weight of 3400 Da was obtained from Nektar Therapeutics (Huntsville, Ala., USA). Oligoethylenimine derivative OEI-HD (20;28;29) is based on OEI 800 Da crosslinked by β-aminoproprionamide linkages and was synthesized by an optimized procedure as described in the following reference: Peter J. Tarcha, Jaroslav Pelisek, Thomas Merdan, Jan Waters, Kent Cheung, K von Gersdorff, Carsten Culmsee, and Ernst Wagner, “Synthesis and Characterization of Chemically Condensed Oligoethyleneimine Containing Beta-Aminopropionamide Linkages for siRNA Delivery,” Biomaterials, 28, 3731 (2007). This protocol generates OEI-HD polymers with a typical molecular weight of 25-30 kDa. All other chemicals were purchased by Sigma-Aldrich (Munich, Germany). Cell culture media, antibiotics and fetal calf serum (FCS) were purchased from Invitrogen (Karlsruhe, Germany). Ready to use siRNA duplexes were purchased from Dharmacon (Lafayette, Colo., USA); LucsiRNA: GL3 luciferase specific duplex: 5′-CWUACGCUGAGUACWUCGAUU-3′, non-specific control siRNA siCONTROL: 5′-UAGCGACUAAACACAUCAAUU-3′ and RAN specific therapeutic siRNA RANsiRNA: 5′-AGAAGAAUCUUCAGUACUAUU-3′ were used.

Cell Culture

For siRNA delivery experiments, mouse neuroblastoma cells Neuro2A (ATCC; CCL-131) and Neuro2A/EGFPLuc cells stably transfected with the EGFPLuc gene (see below) were used. The cells were grown in Dulbecco's modified Eagle's medium (DMEM) supplemented with 10% serum, 4 mM stable glutamine, 100 U/mL penicillin, and 100 μg/mL streptomycin. All cells were seeded 24 hrs prior to siRNA delivery using 5,000 cells per well in 96-well plates for knockdown experiments or applying 200,000 cells in 6-well plates for analysis of protein expression.

Generation of Cells Stably Expressing EGFPLuc Fusion Protein

For the generation of stably transfected cells, the vector pEGFPLuc (Clontech) was linearized using the restriction enzyme Dra III (New England BioLabs, Ipswich, Mass.) and cells were then transfected with this linearized vector using PEI22 (polyethylenimine, 22 kDa, linear; Euromedex, Souffelweyersheim, France). Following gene transfer, Neuro2A cells were incubated in fresh medium for 72 hrs prior to selection of the stably transfected cells with 200 to 2000 μg/mL G418 (Geneticine®; Invitrogen, Karlsruhe, Germany). After several days, surviving cells were seeded at low density into 6-well plates in order to generate separate colonies. Single cell clones were then isolated and expanded. The generated clones were analyzed for the percentage of EGFP-positive (EGFPLuc stably transfected) cells by flow cytometry. Clones with the highest number of EGFP-positive cells were then further selectively grown up under the above described selective conditions and this procedure was repeated until all cells were positive for EGFP. These stably transfected cells expressing approximately 60 ng luciferase per million cells were then used for siRNA delivery experiments.

The synthesis of transferring conjugated OEI-HD (Tf-PEG-OEI) used is described in detail in Example 22.

Formation of Transfection Complexes

Formulations for siRNA delivery were prepared as follows: First, different concentrations of siRNA and OEI-HD were diluted at various polymer/siRNA ratios (w/w: weight/weight) in separate tubes in HBG (HEPES buffered glucose solution; 20 mM HEPES, 5% glucose). Then, the HBG solution of OEI-HD was added to the siRNA, mixed and incubated for 30 to 40 min at room temperature to form stable polymer/siRNA complexes. Transferrin-conjugated OEI-HD/siRNA-formulations were prepared as described above with the exception that OEI-HD polymer was partially replaced with corresponding weight percentages (5, 10, 25, 35, 50%) of Tf-PEG-OEI conjugate, where the weight percentages refer to the OEI component of the Tf conjugate. So replacing 5, 10, or 50% of the OEI-HD with Tf-PEG-conjugated OEI results in transferrin: OEI weight/weight ratios of 0.21/1, 0.41/1 or 2.1/1, respectively, with 17%, 29%, or 68% of the weight of the blended carrier being composed of transferrin protein. For the gene transfer with LF2000/siRNA formulations the manufacturer's standard protocol (Invitrogen) at the ratio 2/1 was applied.

Measurement of Particle Size and Zeta-Potential

Particle size of various polymer/siRNA formulations was measured by dynamic laser-light scattering using a Malvern Zetasizer 3000HS (Malvern Instruments, Worcestershire, UK). For particle sizing complexes were prepared as for gene transfer and diluted in hepes buffered glucose (HBG) to give a final siRNA concentration as used for transfection. For estimation of the zeta-potential, transfection complexes were first prepared in HBG, then 1 mM NaCl was added to achieve appropriate volume for charge measurement and the zeta potential was determined. The data represent the mean of three measurements.

siRNA Delivery into Cells

Experiments for determination of optimal siRNA concentration and polymer/siRNA ratio were performed in stably transfected Neuro2AeGFPLuc cells using LucsiRNA. The cells were seeded in 96-well plates using 5,000 cells per well 24 hrs prior to transfection. For the study with RANsiRNA, Neuro2A cells were used with 4,000 cells per well. Experiments were performed in 80 μL growth medium containing 10% fetal bovine serum (FCS). Transfection complexes for siRNA delivery (20 μL in HBG) were then added to each well without medium change. For protein expression studies, delivery of siRNA was performed in 6-well plates using 200,000 cells per well.

Luciferase Assay

Forty-eight hours following siRNA delivery, medium was removed, cells were washed with phosphate-buffered saline (PBS) and lysed with 50 μL of lysis buffer (25 mM Tris, pH 7.8, 2 mM EDTA, 2 mM dithiothreitol (DTT), 10% glycerol, 1% Triton X-100). Luciferase activity was measured using a Lumat LB9507 instrument (Berthold, Bad Wildbad, Germany). Luciferase light units were recorded from an aliquot of the cell lysate with 10 s integration time after automatic injection of freshly prepared luciferin using the Luciferase Assay system (Promega, Mannheim, Germany). Luciferase activity was measured in triplicates and the relative light units (RLU) were determined.

Cytotoxicity Assay

The cells were grown in 96-well plates and siRNA delivery was performed as described above. Cell viability was determined 1 to 5 days following transfection by addition 10 μL of XTT solution [2,3-bis(2-methoxy-4-nitro-5-sulfophenyl)-2H-tetrazolium-5-carboxyanilide] (Sigma) to each well. Cells were further incubated at 37° C./5% CO₂ for up to two hours and the absorbance at 450 nm was measured spectrophotometrically.

Blood Analysis

Blood was collected by heart puncture immediately after sacrification using heparinized syringes. Counts of white blood cell, red blood cell and platelets were determined immediately following sample collection using a Sysmex XE-2100 whole blood analyser (Sysmex, Norderstedt, Germany). For the determination of various blood enzymes samples were allowed to clot at 37° C. for 4 h, overnight at 4° C., then centrifuged at 3000 g for 20 min at 4° C. and the supernatants were collected for serum analysis. Alkaline and aspartate aminotransaminases (AST, ALT) as well as alkaline phosphatase (AP) were measured using a kinetic UV test from Olympus (Olympus Life and Material Science, Hamburg, Germany). Glutamate Dehydrogenase in plasma was analyzed using a kinetic UV test from Hitado (Hitado Diagnostic Systems, Möhnesee Delecke, Germany).

Histological Examination

Paraffin embedded organs were cut into 5 μm thick sections and stained with hematoxylin and eosin for histopathological examination. Apoptotic cells were determined by TUNEL staining of tissue sections using the ApopTag® Fluorescein kit from Qbiogene (Heidelberg, Germany) according to the manufacturers' protocol. Briefly, deparaffinized tissue sections were treated with 5 mg/mL proteinase K for 15 min at room temperature and inactivated endogenous peroxidase with 3% H₂O₂. Sections were then incubated with TdT enzyme and biotin-labeled and -unlabeled deoxynucleotides at room temperature for 30 min in the dark. Nuclei were counterstained with DAPI.

Western Blot Analysis

For both in vitro and in vivo experiments 50 μg protein per lane was separated by SDS-PAGE under reducing conditions. Proteins were then transferred on a PVDF membrane and blocked with 5% skim milk for one hour at room temperature. Immunostaining was performed using primary Ran antibody (Cell Signaling/Sigma RBI, Taufkirchen, Germany; 1:500) over night at 4° C. according to manufacturer's protocol and peroxidase labelled anti-rabbit-IgG (Cell Signaling; 1:2000) as the secondary antibody for two hours. Ran protein was visualized using ECL western blotting detection system (Upstate). In addition, for the internal correlation of protein expression of each sample, detection of the house keeping protein for α-tubulin was performed using mouse primary antibody (Cell Signaling, 1:5000) for one hour following incubation with anti-mouse HRP-secondary antibody (Cell Signaling, 1:5000) for another hour. For quantification, first Ran protein expression of each sample was normalized for the expression of the house keeping protein for α-tubulin and quantification was performed using the ImageJ System (National Institute of Health, Bethesda, Md., USA).

Statistical Analysis

The siRNA delivery was performed in triplicates with two independent experiments. All values are expressed as means ± standard deviation (SD). Statistical significance of differences was evaluated using Graph Pad Prism 4 software (Graph Pad Software, San Diego, Calif., USA). Experimental groups were compared by Mann-Whitney-U test. The level of significance is indicated with a single asterisk if p≦0.05, two asterisks if p≦0.01, and three asterisks if p≦0.001.

OEI-HD Based siRNA Delivery in Neuro2A-EGFPLuc Cells

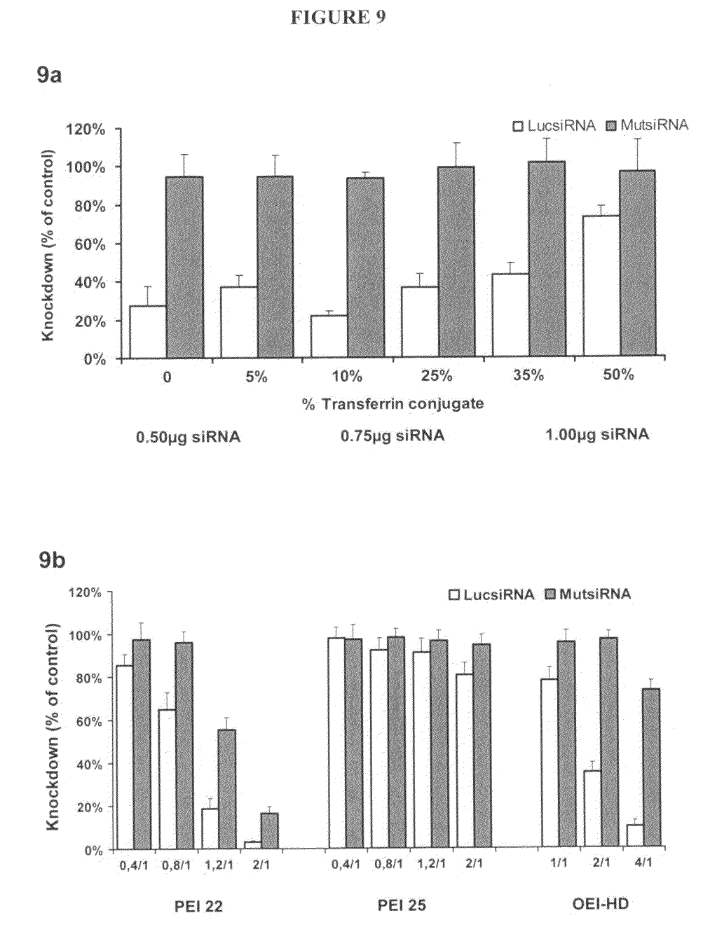

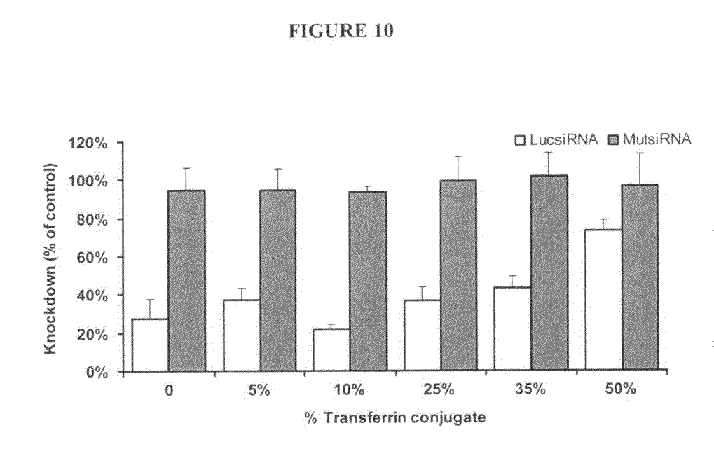

For determination of the optimal transfer conditions to achieve maximal knockdown effect, different amounts of siRNA for up to 1 μg siRNA per 5,000 cells and various polymer/siRNA ratios were tested in stably transfected Neuro2A-EGFPLuc cells constitutively expressing the EGFPLuc fusion protein (FIG. 9A). All formulations were prepared in HBG and siRNA delivery was done in medium containing serum without medium change. At 0.50 μg siRNA per 5,000 cells and further for up to 1 μg siRNA, significant silencing of luciferase expression with up to 80% knockdown was found compared to cells transfected with unspecific siRNA. The optimal OEI-HD/siRNA (w/w) ratio strongly depends on the amount of siRNA used (FIG. 9A). With the increasing amount of siRNA per 5,000 cells the appropriate polymer/siRNA ratio decreased continuously. For 0.5 μg siRNA per 5,000 cells the optimal ratio for maximal knockdown effect was 2/1 (w/w), for 0.75 μg siRNA between 0.8-1/1 and for 1 μg siRNA the most suitable ratio was 0.6-0.8/1. FIG. 10B shows the comparison of 0.25 μg siRNA formulated with different polymers, linear polyethylenimine (PEI22, left), branched polyethylenimine PEI25, center) and OEI-HD (right). Only OEI-HD shows knockdown in the absence of unspecific toxicity. PEI25 shows less than 20% reduction in luciferase activity, PEI22 displays considerable reduction of luciferase activity when also treatment with the mutsiRNA control sequence unspecifically reduces the luciferase activity.

Transferrin-Containing OEI-HD/siRNA Polyplexes

As a next, transferrin (Tf)-containing formulations were tested in Neuro2A-EGFPLuc cells. For this purpose, the OEI-HD polymer was replaced in the formulations with 5% to 50% of Tf-conjugated OEI (FIG. 10). Formulations with up to 35% Tf-conjugate were able to knockdown luciferase activity. Higher amount of Tf conjugates than 35% impaired the silencing effect. The use of 10% of Tf conjugate (i.e. 29 weight % of transferrin in the blended polymer) mediated 80% knockdown in luciferase activity, comparable with the silencing effect of non-targeted particles. In contrast to the non-targeted polyplexes, however, the Tf-based formulations were almost neutral in charge (see below).

Biophysical Properties of OEI-HD/siRNA Polyplexes

Particle size and zeta potential of complexes formed at various polymer (OEI-HD, OEI-HD/OEI-Tf) to siRNA ratios prepared in HBG were determined using dynamic laser-light scattering (Table 1). In all cases, siRNA formulations were overall small-sized in the range of 200-300 nm. Non-targeted OEI-HD/siRNA complexes were the same size of about 200 nm independent of the polymer/siRNA ratio used (0.5/1 to 2/1). Similar results were achieved also for the targeted (10% Tf) OEI-HD/siRNA formulations with the exception of polymer/siRNA ratio 0.5/1 with slightly larger size of about 300 nm.

The zeta-potential of all standard OEI-HD/siRNA formulations had positive charge (Table 1). In correlation with the increasing polymer/siRNA ratio, the charge of the particles increased continuously from +5.7±4.7 mV at the ratio 0.5/1 to +18.6±4.8 mV at the ratio 2/1. In contrast, Tf containing OEI-HD/siRNA formulations showed markedly lower zeta-potential due to transferrin which may serve as a shielding agent. Using 10% Tf and a polymer/siRNA (w/w) ratio of 0.5/1, particles were even slightly negatively charged. At the ratios 0.6/1 and higher (up to 2/1) the Tf-containing formulations were almost neutral. As shown in FIG. 10, using a constant polymer/siRNA ratio of 2/1 and various amounts of Tf between 5-50%, 10% Tf conjugate was sufficient to shield the polyplexes.

TABLE 1
Particle size and zeta potential of OEI/siRNA formulations

| w/w ratio: | | | | |
|---|---|---|---|---|
| 0.5/1 | 0.6/1 | 0.8/1 | 1/1 | 2/1 |

Particle size

| | | | | | |
|---|---|---|---|---|---|
| OEI- | 181 ± 22 | 169 ± 19 | 192 ± 27 | 203 ± 48 | 201 ± 55 |
| HD | | | | | |
| OEI- | 299 ± 72 | 234 ± 21 | 231 ± 48 | 211 ± 37 | 208 ± 43 |
| HD/ | | | | | |
| OEI-Tf | | | | | |

Zeta Potential

| | | | | | |
|---|---|---|---|---|---|
| OEI- | +5.7 ± 4.7 | +6.3 ± 3.4 | +7.8 ± 2.8 | +13.5 ± 2.3 | +18.6 ± 4.8 |
| HD | | | | | |
| OEI- | −6.6 ± 9.7 | +0.5 ± 7.3 | +3.3 ± 4.6 | +5.4 ± 2.2 | +3.6 ± 2.8 |
| HD/ | | | | | |
| OEI-Tf | | | | | |

Formulations were prepared in HBG. For zeta potential, formulations were diluted in 1 mM NaCl prior to measurement.

Cytotoxic Activity of RAN siRNA on Cultured Neuro2A Tumor Cells

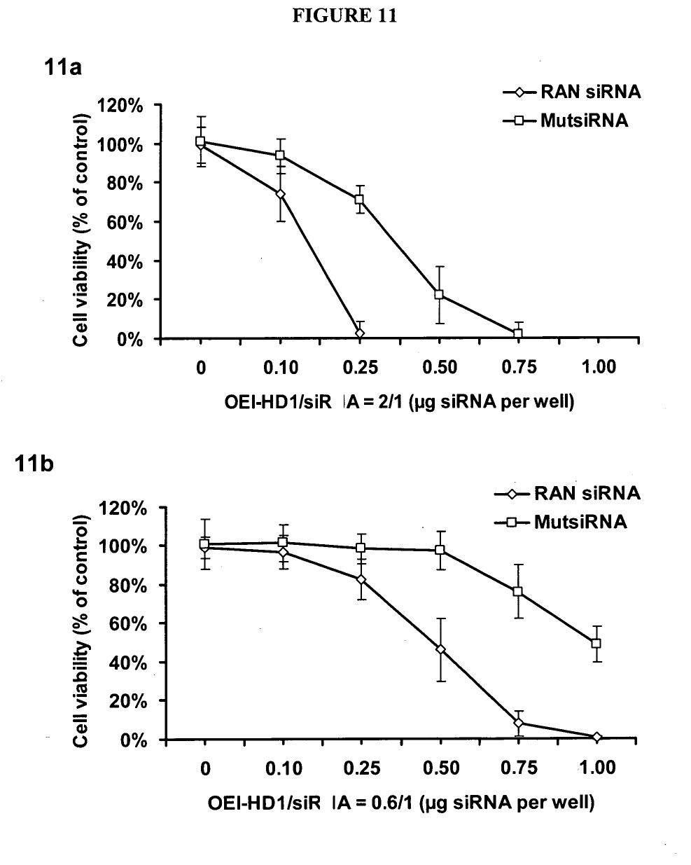

The ras-related nuclear protein Ran was recently identified as interesting anticancer target from an RNAi based screens. We now tested RAN siRNA/OEI-HD polyplexes for antitumoral activity in cultured Neuro2A cells. FIG. 11 shows tumor cell viabilities at day three after polymer/siRNA delivery. Two different polymer/siRNA ratios were used, 0.6/1 (FIG. 11A) and 2/1 (FIG. 11B). In both cases, significant drop down in cell viability was observed compared to application with non-specific siRNA. At the polymer/siRNA ratio 2/1 the maximal cytotoxic activity was observed at 0.25 μg siRNA with up to 80% reduction of cell viability (FIG. 3A). Using the 0.6/1 ratio shows significant cytotoxicity at this dose, complete killing however requires 1 μg siRNA (FIG. 11B).

Knockdown of Ran Protein Expression Following RANsiRNA Application In Vitro

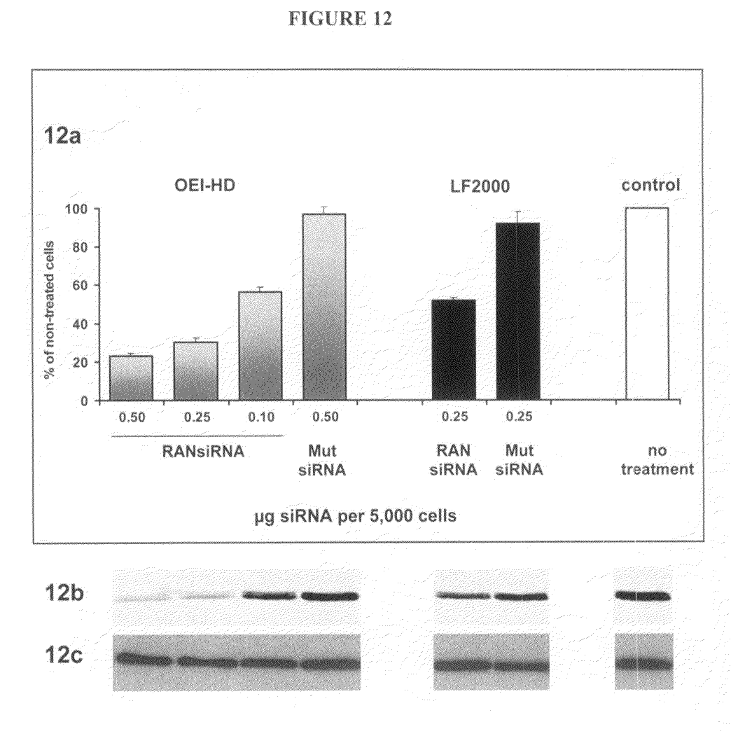

To evaluate the biological effect of RAN siRNA delivery in Neuro2A cells also on the molecular level, western blotting was performed at 3 days after siRNA delivery. Different concentrations of siRNA and the polymer/siRNA ratio of 2/1 were used (FIG. 12). Expression of Ran protein is shown in FIG. 4A by normalizing the RAN expression (FIG. 12B) for the expression of α-tubulin set as 100% (FIG. 12C). Significant reduction in protein expression was observed already using 0.10 μg siRNA per 5,000 cells. At 0.25 μg siRNA per 5,000 cells Ran protein expression was diminished by 70%, and using 0.50-μg siRNA per 5,000 cells 80% reduction of protein expression was found. In contrast, cells treated with unspecific siRNA did not show any significant differences in Ran protein expression compared to non-treated cells. Using LF2000 as a standard siRNA delivery reagent at 0.25 μg siRNA and a LF2000/siRNA ratio of 2/1 per 5,000 cells, only 50% reduction of Ran protein expression was observed.

Reduction of RAN Expression in Neuro2A Tumors after Systemic Delivery of RAN siRNA

Next the in vivo potential of transferrin-targeted OEI-HD based RAN siRNA formulations was tested in a subcutaneous Neuro2A tumor mouse model using Tf-containing OEI-HD/siRNA formulations with 10% Tf conjugate and polymer/siRNA ratio 0.6/1. Treatment by intravenous tail vein injection (35 μg formulated siRNA applied 3 times at 3 days intervals) was started after subcutaneous tumors had reached a size of about 3 mm in diameter. The applied doses were selected based on preliminary dose finding studies using single applications where all (3/3) animals survived the application of 67 μg siRNA formulated with 10% Tf conjugate and polymer/siRNA ratio 0.6/1 (i.e. in total 40 μg of OEI-HD) without any visible side effects or damages of the liver. Free OEI-HD polymer at the 40 μg dose showed significant toxicity and even death in one of three mice as well as liver damage. Therefore in the therapeutic experiments the dosage was reduced to 35 μg siRNA and 21 μg OEI-HD.

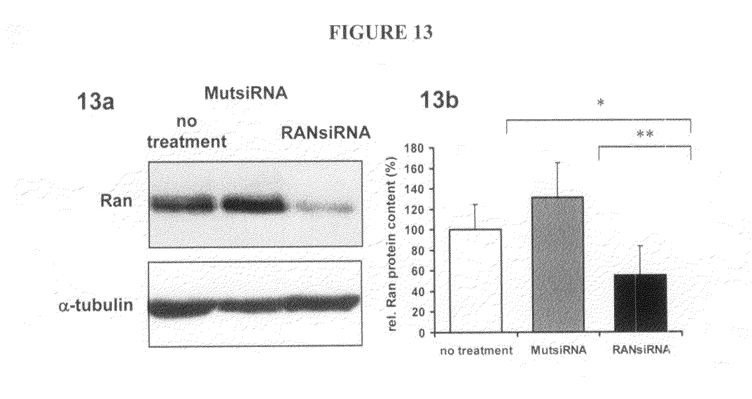

The specific effect of systemically applied RAN siRNA was determined by quantification of Ran protein content in Neuro2A tumors applying western blot analysis 48 hours following the last of three siRNA complex applications and compared with non-specific MutsiRNA and non-treated animals. Using RAN siRNA formulations, an approximately 50% reduced Ran protein expression was observed in tumors as compared to nontreated animals or animals treated with mismatched MutsiRNA (FIG. 13 A-B).

Apoptosis in Tumors of RANsiRNA Treated Mice

In order to analyze the cellular consequences of siRNA-mediated silencing of the RAN gene expression, TdT-mediated dUTP biotin nick-end labeling (TUNEL) analysis of treated tumor tissues was performed, which allows for the detection of apoptotic cells. In order to corroborate the notion that this effect was specific to the tumor cells, liver tissue samples were TUNEL stained and analyzed likewise.

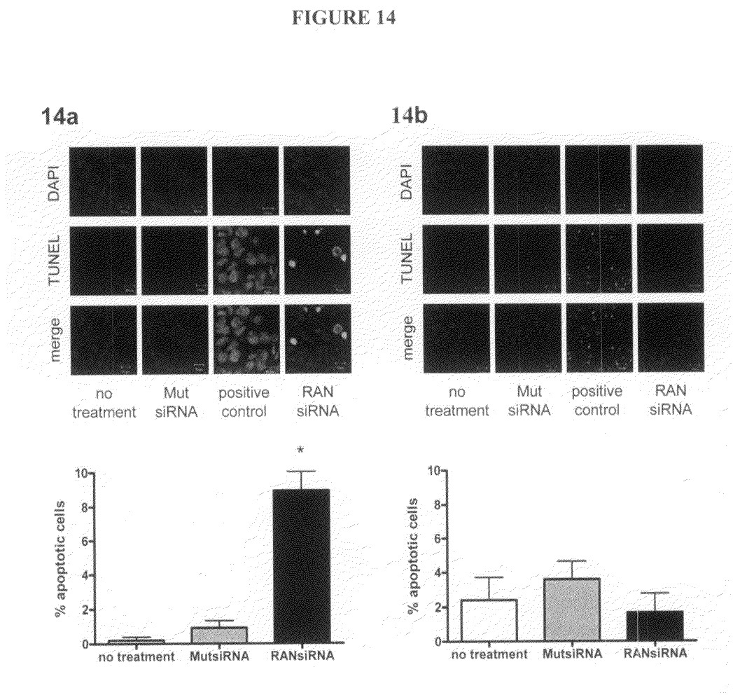

The number of apoptotic cells in the tumors treated with RAN-specific siRNA was increased up to 7-fold compared with non-treated animals (FIG. 14A). Furthermore, non-treated animals and animals treated with the unspecific MutsiRNA showed no significant differences in the amount of apoptotic cells within the tumors. In contrast to the significant increase in apoptosis in RANsiRNA treated tumors, therapeutic siRNA did not markedly affect the liver cells of the same animal (FIG. 14B); no significant differences in the number of apoptotic cells were observed within the liver irrespective of the treatment (specific, non-specific siRNA), as compared to non-treated animals.

Beginning with therapeutic treatment of tumors with a size of about 3 mm in diameter, the tumor volume was measured daily for 7 days. Data representing the tumor growth in the different treatment groups (ten mice per group) did not show convincing differences. In the RAN siRNA application group, a reduction of tumor growth was observed in some animals (data not shown); however it was not statistically significant compared to the control groups, indicating that the clearly observed significant in vivo knockdown and tumor apoptosis was insufficient to lead to a therapeutic effect.

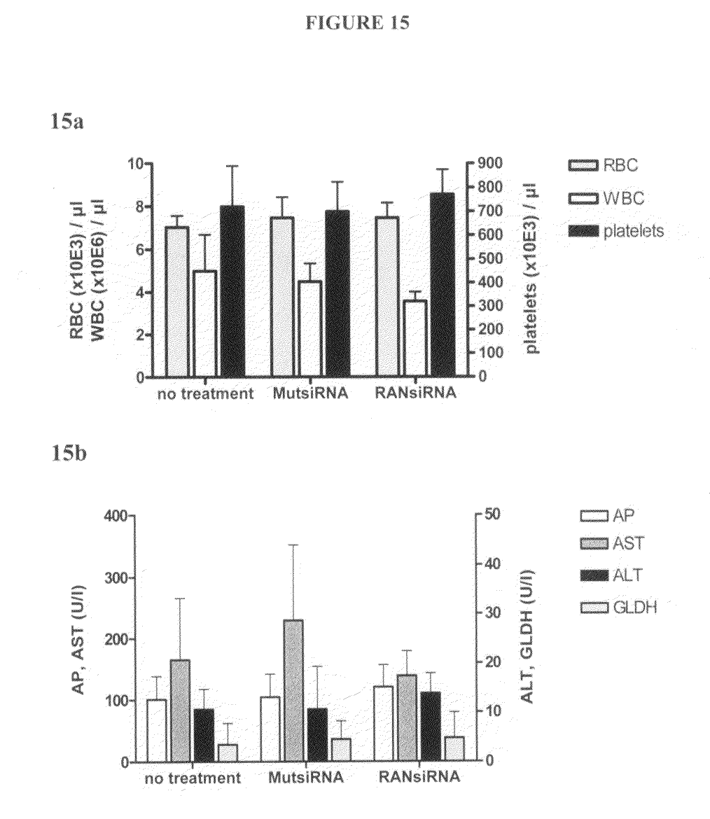

Importantly, no obvious toxicity was observed in the animals during the whole treatment regarding behavior of the animals. Mouse weights were not significantly different among the three groups of animals, suggesting that eating and drinking habits were not affected (data not shown). Additional counting of blood cells and determination of various liver enzymes did not reveal any significant changes in hematology parameters or in the activities of AST, ALT or AP (FIG. 15 A, B).

### Example 19

### In Vivo Use of Chemically Modified Polycation for RAF-1-siRNA Delivery

The experimental protocol described in Example 20 is repeated but substituting SCID-mice, 5 million HuH7 liver tumor cells to generate tumors, and RAF-1-siRNA in the therapy group. RAF-1-siRNA—a small interfering RNA directed against RAF-1, an important biological molecule for many cancer cells.

### Example 20

### In vivo Use of Chemically Modified Polycation for PLK-1—SiRNA Delivery

The experimental protocol described in Example 19 is repeated, but substituting tumor cells that are known to express PLK-1 and have been demonstrated to exhibit cell death after transfection with PLK-1 siRNA in cell culture. PLK-1-siRNA—a small interfering RNA directed against polo-like kinase 1, an important regulator of cell cycle progression. Knockdown of expression leads to toxic effects.

### Example 21

### Formation and Characterization of Polyplexes from Example 19

Characterization of polyplex size is performed using a Malvern Zetasizer Nano ZS. This instrument is capable of measuring Zeta potential by conventional means and particle size by the technique of Quasielastic Laser Light Scattering. OEI-HD, 1.1 micrograms and OEI-PEG-transferrin, 0.12 micrograms are mixed in a total volume of 25 microliters of HBG (HEPES Buffered Glucose, a solution of 5% glucose (weight/volume) containing 20 mM HEPES at a pH of 7.3). The respective amount of siRNA (2.0 micorgrams) was diluted in another vial using HBG to 25 microliters. Subsequently the polymer solution was added to the siRNA solution and mixing was performed by inverting the container 10 times. The resulting polyplex suspension had a particle size between 200-300 nm and a Zeta potential of −1.3 milivolts. Zeta Potential is the electrical potential associated with a colloidal particle moving in an electric field at the surface of shear between the particles stationary ion layer and the mobile ion diffusion layer.

### Example 22

### Coupling of Activated Bifunctional PEG to the Nucleic Acid Carrier OEI-HD-1 Followed by Coupling of Transferrin to the Pendant Activated PEG Endgroups

OEI (34K), 300 mg (˜9×10⁻⁶ mole) were dissolved in 5 ml of 100 mM HEPES buffer, pH 8.6. In a separate vial, 75 mg (66% purity by NMR, ˜1×10⁻⁵ mole) OPSS-PEG(5K)-SPA (ortho-pyridyl disulfide-polyethylene glycol-succinimidyl propionate) were dissolved in 4 ml of ethanol. Both solutions were combined and stirred for 2 hours at room temperature. The reaction solution was subsequently subjected to ion exchange chromatography and the fraction containing the OPSS-PEG-OEI was collected. The volume of the purified product was reduced to 1 ml using a Centricon concentrator. The product was desalted using a PD10 column (pre-saturated w/OEI). This was performed by adding 1 ml of the sample solution to the column followed by addition of 1.5 ml of water. The initial flow-through was discarded and the sample eluted in 2 ml of water. This solution was freeze-dried and analyzed by proton NMR, which indicated one pyridyldithio group per 714 OEI repeat units. This intermediate was named OEI-HD-1-polyethylene glycol-2-pyridyldithio-propionamide or “OEI-PEG-OPSS” for short.

Reduction of OEI-PEG-OPSS to OEI-PEG-SH

25 mg of the freeze-dried OEI-PEG-OPSS were dissolved in 1.5 of 100 mM HEPES buffer. Subsequently, 40 mg (˜2.6×10⁻⁴ mole) of DL-dithiothreitol (DTT) were added into the sample solution and it was stirred for 30 minutes. To monitor the progress of the reaction, 10 μl of the sample solution were diluted to 500 μl with 100 mM HEPES buffer and an absorption scan was performed using an UV/VIS photometer. An observation of a local maximum (343 nm confirmed the progress of this reaction. Using the PD10 column, purified the reaction solution. This was accomplished by adding 1.5 ml of the reaction solution to the column (pre-saturated w/OEI and equilibrated with HBS Buffer) followed by addition of 1 ml of HBS buffer (HBS buffer was bubbled w/argon gas before use). The initial flow-through was discarded and the sample eluted in 2 ml of HBS. The concentration of the OEI-PEG-SH sample solution was 5 mg/ml as determined by a copper complexation assay. For storage, the sample solution was bubbled with argon for five minutes before sealing.

Modification of Transferrin with N-succinimidyl 3-(2-pyridyldithio)-propionate (SPDP)

Transferrin (60 mg) were dissolved in 3 ml of HBS buffer. Subsequently, 3.5 mg of SPDP were dissolved in 7 ml of ethanol (carefully warm the solution for complete dissolution). Immediately 0.5 ml of the SPDP solution were pipetted into the transferrin solution and stirred gently for one hour.

The reaction solution was transferred to a YM-10 Centricon concentrator to reduce the volume to 1 ml. Four more 1 ml buffer exchanges were done on the Centricon concentrator. The purified transferrin-SPDP solution was bubbled with argon gas for five minutes.

Coupling of OEI-PEG-SH with Transferrin-SPDP

10 mg of the OEI-PEG-SH dissolved in 2 ml of HBS buffer solution and 2.7 ml of the purified and activated transferrin solution were united in a new scintillation vial and stirred gently overnight at room temperature. Then the unreacted sulfhydryl groups were quenched with 5 mg (4×10⁻⁵ mole) N-ethyl-maleimide and stirred for 30 minutes.

OEI-PEG-transferrin was purified by ion exchange chromatography. The volume reduced using a Centricon concentrator, and the product desalted as previously described.

The concentration of the polyamine (OEI) in the bioconjugate was 1.3 mg/ml determined by the copper complexation assay and the transferrin assayed at 2.9 mg/ml as determined by UV spectroscopy.

Incorporation of Iron into Transferrin

Iron loading into transferring is done by adding 1.25 μl of the Iron Loading Buffer per milligram of transferrin content into the sample, as described in Kursa M, Walker G F, Roessler V, Ogris M, Roedl W, Kircheis R, Wagner E., Bioconjugate Chem. 2003

The Use of a Shift Assay to Determine the Binding Affinity of siRNA to OEI-PEG 5K-Transferrin.

A siRNA solution of 0.01 mg/ml was prepared from a non-specific siRNA stock solution. Transferred 20 μl of the siRNA solution into a number of small plastic vials, followed by increasing amounts of OEI-PEG 5K-transferrin in HBG buffer solution, keeping the total volume in each vial constant at 40 ul. The weight ratio of OEI-PEG 5K-transferrin-to-siRNA ranged from 0 to 10. After pipetting, the vial contents were mixed well and let stand for 10 minutes. Then 20 μl aliquots from each vial were transferred to their corresponding well in an agarose gel plate. The agarose gel plate was placed in an electrophoresis apparatus set at 75 volts. After 15 minutes the plate was removed and photodocumented. We found that there was no migration band of siRNA observed in the wells of weight ratio 1.5 or higher. This indicates that all siRNA was complexed with OEI(34K)-PEG(5K)-transferrin at weight ratios of 1.5 and higher.

### Example 23

### Coupling of Benzoylbenzoic Acid to Compounds of Formula I or Ia

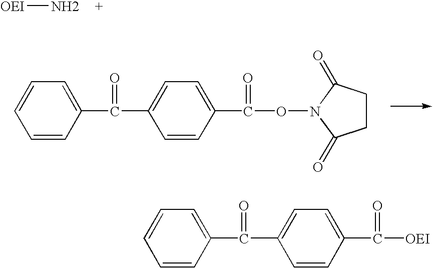

(RLU=relative luminescence units; C:P+ carrier to plasmid or siRNA ration w/w; luc=anti-luciferase siRNA; scr=scrambler siRNA control; and lip=lipofectamine transfection agent.)

Benzoiylbenzoic acid-succinimidyl ester (Invitrogen Cat. No. 1577) was used and the reaction was performed in HBS (Hepes buffered Saline, 10 mM HEPES, 150 mM NaCl, pH 7.3,). 20 mg polymer (described below) were dissolved in 2 ml HBS and pH was adjusted to be 7.3. Subsequently different amounts of benzoylbenzoic acid-NHS ester were added in 1 ml anhydrous DMSO.

All reactions were run for 12 hours before dilution to approximately 6 ml was performed using deionised water. Subsequently dialysis was performed and after that conjugates were freeze-dried.

TABLE 2
Dialysis membranes used:

| Basal Polymer | Membrane MWCO |
|---|---|
| PEI 2 kDa | 1 kDa |
| OEI 5 kDa | 3 kDa |
| OEI 30 kDa | 10 kDa |

TABLE 3
Actual degrees of coupling for the conjugates synthesized

| | Benzoulbenzoic acid |
|---|---|
| Conjugate | per polymer (NMR) |
| PEI 2 kDa low | 2.5 |
| PEI 2 kDa medium | 7 |
| PEI 2 kDa high | 15 |
| OEI 5 kDa low | 1.8 |
| OEI 5 kDa medium | 12 |
| OEI 5 kDa high | 20 |
| OEI 30 kDa low | 1 |
| OEI 30 kDa medium | 3 |
| OEI 30 kDa high | 8 |

The samples were freeze-dried again and dissolved in HBS (Hepes buffered Saline, 10 mM HEPES, 150 mM NaCl, pH 7.3). From these solutions concentrations were determined against unmodified polymer using the copper assay.

### Size Determinations Via Dynamic Laser Light Scattering

0.5 microg of siRNA were complexed with the respective amount of OEI in a total volume of 50 microliters.

TABLE 4

| | PEI 2K | PEI 2K | PEI 2K |
|---|---|---|---|
| C:P | benzo-1 | benzo-2 | benzo-3 |
| 0.3 | 254 | 370 | 392 |
| 0.6 | 202 | 239 | 204 |
| 1.2 | 178 | 198 | 194 |
| 2.0 | 182 | 195 | 189 |

TABLE 5

| | OEI5K | OEI5K | OEI5K |
|---|---|---|---|
| C:P | benzo-1 | benzo-2 | benzo-3 |
| 0.3 | 370 | 72 | mult |
| 0.6 | 303 | 350 | 195 |
| 1.2 | 196 | 210 | 260 |
| 2.0 | 190 | 121 | 99 |

mult = multiple peaks

TABLE 6

| | OEI30K | OEI30K | OEI30K |
|---|---|---|---|
| C:P | benzo-1 | benzo-2 | benzo-3 |
| 0.3 | 436 | 457 | 342 |
| 0.6 | 151 | mult | 249 |
| 1.2 | mult | mult | 116 |
| 2.0 | mult | mult | 120 |

### Example 24

### Coupling of lauric acid to Compounds of Formula I or Ia

Lauric acid N-hydroxy-succinimidyl ester (Sigma-Aldrich # OL3900-5g, Lot # 087H5174) was used and the reaction was performed in HBS (Hepes buffered Saline, 10 mM HEPES, 150 mM NaCl, pH 7.3).

20 mg polymer were dissolved in 2 ml HBS and pH was adjusted to be 7.3. Subsequently different amounts of lauric acid NHS ester were added in 1 ml anhydrous DMSO.

TABLE 12
PEI 800 Da and PEI 2 kDa

| | | mg lauric | | |
|---|---|---|---|---|
| PEI 800 Da | PEI 2 kDa | acid NHS | acid per PEI | Remarks |
| 20 mg | — | 7.4 | 1 | none |
| 20 mg | — | 29.7 | 4 | precipitation |
| 20 mg | — | 59.4 | 8 | precipitation |
| — | 20 mg | 5.9 | 2 | none |
| — | 20 mg | 23.8 | 8 | precipitation |
| — | 20 mg | 35.6 | 12 | precipitation |

TABLE 13
OEI 5 kDa, 9 kDa and 30 kDa

| 20 mg | mg lauric acid NHS | acid per PEI | Remarks |
|---|---|---|---|
| 5 kDa | 2.4 | 2 | None |
| 5 kDa | 11.9 | 10 | None |
| 5 kDa | 29.7 | 25 | cloudiness |
| 9 kDa | 2.7 | 4 | None |
| 9 kDa | 10 | 15 | None |
| 9 kDa | 27 | 40 | precipitation |
| 30 kDa | 1.2 | 6 | None |
| 30 kDa | 5 | 25 | None |
| 30 kDa | 20 | 100 | precipitation |

All reactions were run for 12 hours before dilution to approximately 6 ml was performed using deionised water. Subsequently dialysis was performed and after that conjugates were freeze-dried.

TABLE 14

| Conjugate basal | Dialysis Membrane |
|---|---|
| polymer | cut off |
| PEI 800 | 500 |
| PEI 2 kDa | 1 kDa |
| OEI 5 kDa | 3 kDa |
| OEI 9 kDa | 3 kDa |
| OEI 30 kDa | 10 kDa |

### Subsequently the conjugates were dissolved in D2O and submitted for ¹H-NMR.

### The following actual coupling degrees were determined via NMR.

Coupling degrees based on NMR calculations:

TABLE 15

| Conjugate | Lauric Acid Per Polymer |
|---|---|
| PEI 800 | Could not be processed due to cloudiness |
| PEI 2k | 0.5 |
| OEI 5k low | 1.6 |
| OEI 5k medium | 8 |
| OEI 5k high | Could not be processed due to low solubility |
| OEI 9k low | 4 |
| OEI 9k medium | 12 |
| OEI 30k low | 4 |
| OEI 30k medium | 22 |

### The samples were freeze-dried again and dissolved in HBS (Hepes buffered Saline, 10 mM HEPES, 150 mM NaCl, pH 7.3).

### From these solutions concentrations were determined against unmodified polymer using the copper assay.

Size Determinations Via Dynamic Laser Light Scattering

0.5 microg of siRNA were complexed with the respective amount of OEI in a total volume of 50 microliters. Experiments were carried out in HBS.

TABLE 16
SIZE

| C:P | PEI 800 | PEI2K | PEI2K-LA |
|---|---|---|---|
| 0.3 | 297 | 116 | 136 |
| 0.6 | 250 | mult | 113 |
| 1.2 | 215 | mult | 121 |
| 2.0 | 192 | mult | 123 |

TABLE 17
SIZE

| C:P | OEI5K | OEI5K LA1 | OEI5K LA2 |
|---|---|---|---|
| 0.3 | mult | 83 | 76 |
| 0.0 | mult | mult | 72 |
| 1.2 | mult | mult | mult |
| 2.0 | mult | mult | mult |

TABLE 18
SIZE

| C:P | OEI9K LA-1 | OEI5K LA-2 |
|---|---|---|
| 0.3 | 162 | 88 |
| 0.6 | 124 | 80 |
| 1.2 | mult | 88 |
| 2.0 | mult | mult |

### Plain OEI 9k gave multiple peaks over the whole range.

TABLE 19
SIZE

| C:P | OEI30K LA-1 | OEI30K LA-2 |
|---|---|---|
| 0.3 | 108 | 94 |
| 0.6 | mult | mult |
| 1.2 | mult | mult |
| 2.0 | mult | mult |

### Knockdown efficiency H1299 cells, 0.5 microg siRNA per well.

PEI 800 and PEI2k and Derivatives Thereof

### Example 25

### Coupling of lauric acid to PEIs/OEIs and physicochemical as well as biological evaluation of conjugates

Lauric acid N-hydroxy-succinimidyl ester (Sigma-Aldrich # OL3900-5g, Lot # 087H5174) was used and the reaction was performed in HBS (Hepes buffered Saline, 10 mM HEPES, 150 mM NaCl, pH 7.3).

20 mg polymer were dissolved in 2 ml HBS and pH was adjusted to be 7.3. Subsequently different amounts of lauric acid NHS ester were added in 1 ml anhydrous DMSO. PEI 800 Da and PEI 2 kDa

TABLE 30

| PEI | PEI | mg lauric | Acid per | |
|---|---|---|---|---|
| 800 Da | 2 kDA | acid NHS | PEI | Remarks |
| 20 mg | — | 7.4 | 1 | None |
| 20 mg | — | 29.7 | 4 | Precipitation |
| 20 mg | — | 59.4 | 8 | Precipitation |
| — | 20 mg | 5.9 | 2 | None |
| — | 20 mg | 23.8 | 8 | Precipitation |
| — | 20 mg | 35.6 | 12 | Precipitation |

### OEI 5 kDa, 9 kDa and 30 kDa

TABLE 31

| 20 mg | mg lauric acid NHS | Acid per PEI | Remarks |
|---|---|---|---|
| 5 kDa | 2.4 | 2 | None |
| 5 kDa | 11.9 | 10 | None |
| 5 kDa | 29.7 | 25 | Cloudiness |
| 9 kDa | 2.7 | 4 | None |
| 9 kDa | 10 | 15 | None |
| 9 kDa | 27 | 40 | Precipitation |
| 30 kDa | 1.2 | 6 | None |
| 30 kDa | 5 | 25 | None |
| 30 kDa | 20 | 100 | Precipitation |

All reactions were run for 12 hours before dilution to approximately 6 ml was performed using deionised water. Subsequently dialysis was performed and after that conjugates were freeze-dried.

TABLE 32

| Conjugate basal | Dialysis Membrane |
|---|---|
| polymer | cut off |
| PEI 800 | 500 |
| PEI 2 kDa | 1 kDa |
| OEI 5 kDa | 3 kDa |
| OEI 9 kDa | 3 kDa |
| OEI 30 kDa | 10 kDa |

### Subsequently the conjugates were dissolved in D₂O and submitted for ¹H-NMR.

### The following actual coupling degrees were determined via NMR.

Coupling degrees based on NMR calculations:

TABLE 33

| Conjugate | Lauric Acid per Polymer |
|---|---|
| PEI 800 | Could not be processed due to cloudiness |
| PEI 2k | 0.5 |
| OEI 5k low | 1.6 |
| OEI 5k medium | 8 |
| OEI 5k high | Could not be processed due to low solubility |
| OEI 9k low | 4 |
| OEI 9k medium | 12 |
| OEI 30k low | 4 |
| OEI 30k medium | 22 |

The samples were freeze-dried again and dissolved in HBS (Hepes buffered Saline, 10 mM HEPES, 150 mM NaCl, pH 7.3.

From these solutions concentrations were determined against unmodified polymer using the copper assay.

### Size Determinations Via Dynamic Laser Light Scattering

0.5 microg of siRNA were complexed with the respective amount of OEI in a total volume of 50 microliters. Experiments were carried out in HBS.

TABLE 34
SIZE

| C:P | PEI 800 | PEI2K | PEI2K-LA |
|---|---|---|---|
| 0.3 | 297 | 116 | 136 |
| 0.0 | 250 | mult | 113 |
| 1.2 | 215 | mult | 121 |
| 2.0 | 192 | mult | 123 |

TABLE 35
SIZE

| C:P | OEI5K | OEI5K LA1 | OEI5K LA2 |
|---|---|---|---|
| 0.3 | mult | 83 | 76 |
| 0.0 | mult | mult | 72 |
| 1.2 | mult | mult | mult |
| 2.0 | mult | mult | mult |

TABLE 36
SIZE

| C:P | OEI9K LA-1 | OEI5K LA-2 |
|---|---|---|
| 0.3 | 162 | 88 |
| 0.6 | 124 | 80 |
| 1.2 | mult | 88 |
| 2.0 | mult | mult |

TABLE 37
SIZE

| C:P | OEI30K LA-1 | OEI30K LA-2 |
|---|---|---|
| 0.3 | 108 | 94 |
| 0.6 | mult | mult |
| 1.2 | mult | mult |
| 2.0 | mult | mult |

### Knockdown efficiency H1299 cells, 0.5 microg siRNA per.

PEI 800 and PEI2k and Derivatives Thereof

### Example 26

### Coupling of Benzoylbenzoic Acid to PEIs/OEIs and Physicochemical as Well as Biological Evaluation of Conjugates

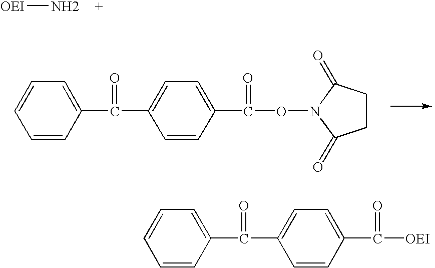

Benzoiylbenzoic acid-succinimidyl ester (Invitrogen # 1577) was used and the reaction was performed in HBS (Hepes buffered Saline, 10 mM HEPES, 150 mM NaCl, pH 7.3). 20 mg polymer were dissolved in 2 ml HBS and pH was adjusted to be 7.3. Subsequently different amounts of benzoylbenzoic acid-NHS ester were added in 1 ml anhydrous DMSO. All reactions were run for 12 hours before dilution to approximately 6 ml was performed using deionised water. Subsequently dialysis was performed and after that conjugates were freeze-dried.

TABLE 48

| | benzoylbenzoic acid | |
|---|---|---|
| 20 mg polymer | NHS ester [mg] | acid per polymer |
| PEI 2 kDa | 3.2 | 1 |
| PEI 2 kDa | 10 | 3 |
| PEI 2 kDa | 19 | 6 |
| OEI 5 kDa | 2.55 | 2 |
| OEI 5 kDa | 12.9 | 10 |
| OEI 5 kDa | 26 | 20 |
| OEI 30 kDa | 1.3 | 6 |
| OEI 30 kDa | 4.3 | 20 |
| OEI 30 kDa | 10.8 | 50 |

Dialysis membranes used:

TABLE 49

| Basal Polymer | Membrane MWCO |
|---|---|
| PEI 2 kDa | 1 kDa |
| OEI 5 kDa | 3 kDa |
| OEI 30 kDa | 10 kDa |

### Actual degrees of coupling for the conjugates synthesized

TABLE 50

| | Benzoulbenzoic acid |
|---|---|
| Conjugate | per polymer (NMR) |
| PEI 2 kDa low | 2.5 |
| PEI 2 kDa medium | 7 |
| PEI 2 kDa high | 15 |
| OEI 5 kDa low | 1.8 |
| OEI 5 kDa medium | 12 |
| OEI 5 kDa high | 20 |
| OEI 30 kDa low | 1 |
| OEI 30 kDa medium | 3 |
| OEI 30 kDa high | 8 |

The samples were freeze-dried again and dissolved in HBS (Hepes buffered Saline, 10 mM HEPES, 150 mM NaCl, pH 7.3).

### From these solutions concentrations were determined against unmodified polymer using the copper assay.

### Size Determinations Via Dynamic Laser Light Scattering

0.5 microg of siRNA were complexed with the respective amount of OEI in a total volume of 50 microliters.

TABLE 51

| | PEI 2K benzo-1 | PEI 2K benzo-2 | PEI 2K |
|---|---|---|---|
| C:P | low | medium | benzo-3 high |
| 0.3 | 354 | 370 | 392 |
| 0.0 | 202 | 239 | 204 |
| 1.2 | 178 | 198 | 194 |
| 2.0 | 182 | 195 | 189 |

TABLE 52

| C:P | OEI 5K benzo-1 | OEI 5K benzo-2 | OEI 5K benzo-3 |
|---|---|---|---|
| 0.3 | 370 | 72 | mult |
| 0.0 | 303 | 350 | 195 |
| 1.2 | 196 | 210 | 260 |
| 2.0 | 190 | 121 | 99 |

mult = multiple peaks

TABLE 53

| C:P | OEI 30K benzo-1 | OEI 30K benzo-2 | OEI 30K benzo-3 |
|---|---|---|---|
| 0.3 | 436 | 457 | 342 |
| 0.0 | 151 | mult | 249 |
| 1.2 | mult | mult | 116 |
| 2.0 | mult | mult | 120 |

Knockdown efficiency H1299 cells, 0.5 microg siRNA per well.

What is claimed is:

1. A polymer formed by polycations chemically linked by propionylamide units, wherein said polymer is useful as a non-viral carrier for nucleic acid delivery.

2. The polymer of claim 1 wherein the polycation is polyethyleneimine (PEI).

3. The polymer of claim 1 that is useful for siRNA delivery.

4. The polymer of claim 1 further comprising a shielding ligand.

5. The polymer of claim 4, wherein the shielding ligand is polyethylene glycol (PEG).

6. The polymer of claim 5 further comprising a targeting ligand.

7. The polymer of claim 6 wherein the targeting ligand is transferrin.

8. The polymer of claim 4 further comprising coupling with a polynucleotide.

9. The polymer of claim 5 further comprising coupling with a polynucleotide.

10. The polymer of claim 6 further comprising coupling with a polynucleotide.

11. The polymer of claim 7 further comprising coupling with a polynucleotide.

12. A method of making the polymer of claim 1, wherein the polymer is cross-linked by the Michael addition of a fraction of the polymer's amines to vinylic groups of cross-linking agent and further modified by N-acylation of pendant ester groups.

13. The method of claim 12 wherein the polymer is further modified by addition of free ester, anhydryde, or acylhalide.

14. The method of claim 12 wherein the cross-linking can occur at both the primary and secondary amines of the polymer structure.

15. The method of claim 12 wherein the cross-linking agents comprise ester monomers of the following agents: acrylate, methacrylate, ethylene glycol diacrylate, ethylene glycol dimethacrylate, 1,6 hexanediol diacrylate, and polyethylene glycol 600 diacrylate.

16. The method of claim 15 wherein the cross linking agent is 1,6. hexanediol diacrylate.

17. A method of delivering genes to target tissue in vivo using the polymer of claim 1.

18. A method of delivering siRNA to cells in culture using the polymer of claim 1.

19. A method of delivering siRNA to tissue in vivo using the polymer of claim 1.

20. The method of claim 19, wherein the delivery of siRNA is for therapeutic purposes or for target validation.

21. A method of using the polymer of claim 1, wherein the polymer forms a targetable complexing agent that can bind entities of opposite charge and delivery them to target tissue.

22. A method of delivering therapeutic entity of interest to a patient using the polymer of claim 1, wherein the polymer forms a ionic complex or covalent bond with the therapeutic entity of interest.

23. The method of claim 22, wherein the therapeutic entity of interest comprises cytotoxic agents, endosomal lytic agents, and hydrophilic polymers.

24. A compound of Formula I comprising:
[[polycation]ₐ-[L]_{b}-[polycation]_{c}]_{d}
wherein:
polycation is a polyethyleneimine unit;
L is a non-ester linker;
a is an integer in the range of about 1 to about 20;
b is an integer in the range of about 1 to about 10;
c is an integer in the range of about 1 to about 20; and
d is an integer in the range of about 1 to about 1000.

25. The compound of claim 24 in which the polycation is oliogethyleneimine.

26. The compound of claim 24 in which L is selected from the group consisting of an amido linker moiety, an amino linker moiety, a carbonyl linker moiety, a carbamate linker moiety, a urea linker moiety, an ether linker moiety, a disulphide linker moiety, and a succinamidyl linker moiety.

27. The non-ester linker of claim 26 in which L is a beta-aminopropionylamide linker moiety.

28. The compound of claim 24 further comprising a biomolecule.

29. The compound of claim 28 wherein said biomolecule is siRNA.

30. The compound of claim 24 having a weight average molecular weight in the range of about 800 Daltons to about 1,000,000 Daltons.

31. The compound of claim 24 having a weight average molecular weight in the range of about 20,000 Daltons to about 200,000 Daltons.

32. The compound of claim 24 having a weight average molecular weight in the range of about 20,000 Daltons to about 30,000 Daltons.

33. A compound of Formula I comprising: 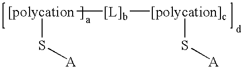
wherein:
polycation is a polyethylenimine;
L a non-ester linker;
S is a spacer or is absent;
A is an agent or is absent;
a is an integer in the range of about 1 to about 20;
b is an integer in the range of about 1 to about 10;
c is an integer in the range of about 1 to about 20; and
d is an integer in the range of about 1 to about 1000.

34. The compound of claim 33 in which the polycation is oliogethyleneimine.

35. The compound of claim 33 in which L is selected from the group consisting of an amido linker moiety, an amino linker moiety, a carbonyl linker moiety, a carbamate linker moiety, a urea linker moiety, an ether linker moiety, a disulphide linker moiety, and a succinamidyl linker moiety.

36. The non-ester linker of claim 33 in which L is a beta-aminopropionylamide linker moiety.

37. The compound of claim 33 further comprising a biomolecule.

38. The compound of claim 37 wherein said biomolecule is siRNA.

39. The compound of claim 33 further comprising an agent and optionally S.

40. The compound of claims 38 or 39 wherein said agent is selected form the group consisting of an agent that facilitates receptor recognition, internalization, escape of the biomolecule from cell endosome, nucleus localization, biomolecule release, and system stabilization.

41. The compound of claims 38 or 39 wherein said agent is selected from the group consisting of a cytotoxic agent, a hydrophobic group, a shielding agent, and a targeting ligand.

42. The compound of claim 41 wherein said agent is transferrin.

43. A non-viral delivery system comprising: (a) a biomolecule; (b) a compound coupled to the biomolecule, wherein the compound-biomolecule conjugate comprises the compound of claims 24 or 33.

44. A non-viral delivery system of claim 45 wherein said biomolecule is siRNA

45. A method of treating a mammal, comprising identifying a mammal in need of gene therapy and administering the compound of claim 28 to the mammal, wherein said biomolecule is siRNA that is effective to lower expression of a gene of interest.

46. A method of treating a mammal, comprising identifying a mammal in need of gene therapy and administering the compound of claim 28 to the mammal, wherein said biomolecule is siRNA that is effective to lower expression of a gene of interest.
